(19) United States
(12) Patent Application Publication
(10) Pub. No.: US 2015/0104440 A1
(43) Pub. Date: Apr. 16, 2015

(54) MiRNA-31 AS A DIAGNOSTIC, PROGNOSTIC AND THERAPEUTIC AGENT IN CANCER

(71) Applicant: CORNELL UNIVERSITY, Ithaca, NY (US)

(72) Inventors: Pei-Chun Lin, New York, NY (US); Mark A. Rubin, New York, NY (US)

(73) Assignee: CORNELL UNIVERSITY, Ithaca, NY (US)

(21) Appl. No.: 14/381,394

(22) PCT Filed: Apr. 12, 2013
(86) PCT No.: PCT/US2013/036300

§ 371 Date: Aug. 27, 2014

Related U.S. Application Data
(60) Provisional application No. 61/623,266, filed on Apr. 12, 2012.

(51) Int. Cl. C12Q 1/68; C12N 15/113

(52) U.S. Cl. CPC C12Q 1/6886; C12N 15/113; C12Q 2600/154; C12Q 2600/158; C12Q 2600/178; C12N 2310/141
USPC 424/130.1; 435/6.11; 435/6.12; 506/12; 514/44 A; 514/19.5; 536/24.5

(57) ABSTRACT

The current disclosure reveals a complex regulatory pattern between miR-31 and AR, indicating that miR-31 plays a key role in prostate cancer development and progression. Another aspect of the current disclosure shows that miR-31 directly targets and destabilizes AR mRNA through interaction with the AR mRNA coding sequence showing that miR-31, or a fragment thereof has the ability to act as a novel therapeutic agent in treating cancer. The current disclosure also shows that AR indirectly represses miR-31 expression by binding to the miR-31 promoter region and modulating methyltransferase activity. Another aspect of the current disclosure shows that miR-31 indirectly modulates AR activity by modulating regulators of cell cycle progression. The disclosure further provides an isolated nucleic acid that modulates the activity of the androgen receptor in a cell. The disclosure further provides a method of treating a prostate cancer in a subject, by administering to the subject an effective amount of an agent that modulates the activity or levels of miR-31.

### CROSS REFERENCE TO RELATED APPLICATIONS

This application claims priority from U.S. Provisional Application No. 61/623,266, filed Apr. 12, 2012, the entire contents of which are incorporated herein by reference.

### GOVERNMENT SUPPORT

The present disclosure was supported with government support under grant number CA 11275-07 awarded by the National Institutes of Health. The government has certain rights in the disclosure.

### FIELD OF THE DISCLOSURE

The present disclosure relates to compositions and methods concerning the identification, treatment and characterization cancer, as well as use of microRNAs (miRNAs) related to such, for therapeutic, prognostic, and diagnostic applications, particularly those methods and compositions related to assessing and/or identifying prostate cancer, directly or indirectly related to microRNA-31 (miR-31) or androgen receptor (AR) activity and/or expression.

### BACKGROUND OF THE DISCLOSURE

miRNAs are small, non-coding single-stranded RNAs with predicted potential to regulate over 30% of the human protein coding genes at the post-transcriptional level, mainly by binding to the 3′-UTR of their mRNA targets (see Bartel D P, Cell. (2004) 116: 281-297; Lewis B P et al. Cell. (2005), 120: 15-20). Numerous studies in recent years have shown that miRNAs play important roles in multiple biological processes, such as development and differentiation, cell proliferation, apoptosis, metabolism, and stress response (see Alvarez-Garcia I et al., Development. (2005), 132: 4653-4662; Cheng A M et al. Nucleic Acids Res. (2005), 33: 1290-1297).

Many of these processes are often perturbed in cancer. Some miRNAs have been identified acting as either oncogenes or tumor suppressors (See, e.g., Croce C M. Nat Rev Genet. (2009), 10:704-14; Jeronimo, C, et al., European Urology. (2011), 60:753-766; Calin G A et al., Proc Natl Acad Sci USA. (2002); 99: 15524-15529; Takamizawa J et al., Cancer Res. (2004); 64: 3753-3756).

Prostate cancer (PCA) represents a major public health problem among the aging Western population. It has the highest incidence rate of all noncutaneous malignancies in men, accounting for more than 241,000 new cases and 28,000 deaths in the United States in 2012 (see Siegel R, et al., CA Cancer J Clin. (2012), 62:10-29). PCA depends largely on androgen receptor (AR) signaling for growth and maintenance. Following the seminal observations by Huggins and Hodges over 60 years ago that PCA responded dramatically to castration, androgen deprivation therapy (ADT) has become the standard first-line treatment for advanced hormone naïve PCA (see Messing E M, et al., N Engl J Med. (1999); 341:1781-88; Huggins C, et al., Arch Surg. (1941), 43:209-15). By reducing circulating androgen, ADT prevents signaling through AR and limits cancer growth. Unfortunately, the beneficial effect of ADT is short-lived and patients progress to castration-resistant prostate cancer (CRPC). While these observations have led to the development of more efficacious therapeutic approaches for targeting AR signaling (see e.g. Chen Y., et al., Curr. Opin. Pharmacol. (2008) 8(4):440-448), CRPC still persists after treatment; therefore, other interventions are needed for AR regulation.

Epigenetic aberrations arise during PCA initiation and disease progression, which include promoter cytosine-guanine (CpG) island hypermethylation at specific gene loci and changes in chromatin structure (see Jones P A, et al., Nat Rev Genet. (2002), 3:415-28). As stated above, miRNAs are involved in critical cellular functions in a tissue-specific manner, aberrant expression of miRNAs can contribute to tumorigenesis by inducing oncogenes, inhibiting tumor suppressor genes, or disrupting important signaling pathways (see Croce, C M. (2009)). To date, little is known about the association between DNA methylation, miRNA expression, and AR signaling.

### SUMMARY OF THE DISCLOSURE

The present disclosure provides a method of diagnosing prostate cancer in a subject wherein a biological sample is obtained from the subject and the level of miR-31 promoter methylation is measured, wherein an alteration in miR-31 promoter methylation is indicative of whether or not prostate cancer exists in the subject. In one embodiment, an alteration is determined by comparing the level of miR-31 promoter methylation in a test sample to a control, such as that of a sample obtained from benign tissue, including but not limited to benign prostate tissue. In another embodiment, the level of miR-31 promoter methylation is used to determine the severity of prostate cancer in the subject.

The disclosure further provides a method of diagnosing prostate cancer in a subject by determining the level of expression of miR-31, wherein a biological sample is obtained from the subject and the level of miR-31 expression is measured, wherein the level of miR-31 expression indicates the presence of prostate cancer in the subject. In one embodiment, the level of miR-31 expression in the sample is compared to a control, such as that of a sample obtained from benign tissue, including but not limited to benign prostate tissue, and a decreased level of miR-31 expression in the sample relative to that of benign tissue indicates the presence of prostate cancer in the subject. In another embodiment of the current disclosure, the level of miR-31 expression indicates the severity of prostate cancer in the subject.

The disclosure further provides a method of determining whether a subject having prostate cancer is a candidate for treatment with a therapeutic agent. Wherein a biological sample is obtained from a subject, and the level of miR-31 promoter methylation is measured, and the subject is rejected as a candidate for treatment when the level of miR-31 promoter methylation is decreased relative to a control, and the subject may be selected as a suitable candidate for treatment if miR-31 promoter methylation is elevated as compared to a control.

The disclosure further provides a method of treating a prostate cancer in a subject, by administering to the subject an effective amount of an agent that modulates the activity of miR-31. In one embodiment of the current disclosure the agent directly or indirectly modulates the expression of miR-31, modulates miR-31 promoter methylation, or modulates the interaction between miR-31 and a regulator of cell cycle progression. In yet another embodiment a second therapeutic agent is provided to the subject including administration of a chemotherapeutic agent, radiation or an AR targeting therapeutic agent.

The disclosure further provides an isolated nucleic acid that inhibits the activity of AR in a cell. In a further embodiment, the isolated nucleic acid is miR-31 or a fragment thereof

These and other embodiments of the disclosure will be readily apparent to those of ordinary skill in view of the disclosure herein.

### BRIEF DESCRIPTION OF DRAWINGS AND TABLES

FIG. 1 shows MiR-31 is downregulated in PCA due to promoter hypermethylation. (A) Heatmap of the 25 differentially expressed miRNAs in PCA as compared to matched benign tissues (Benign), red=high expression, green=low expression. (B) Expression ratio of miR-31 in PCA to matched Benign, red line for ratio 1. (C) Expression of miR-31 and MIR31HG in 40 PCA and 15 Benign as evaluated by qPCR. (D) Deletion analysis of chromosome region 9p21.3 in various cancer types, gray indicates genes that fall within the deletion peak. (E) DNA methylation levels at the miR-31 promoter in PCA (n=12) and Benign (n=12). (F) Graph illustration showing genomic organization of MIR31HG, the relative location of miR-31 and the CpG-island, and the positions of four regions PCR amplified by four sets of primers as well as the representative locations of CpG units within the regions. (G) DNA methylation at the miR-31 promoter in indicated cell lines. Top: comparison of overall DNA methylation levels; bottom: heatmap of DNA methylation levels. Each row corresponds to an individual sample, and each column corresponds to an individual CpG unit, which is a single CpG site or a combination of CpG sites. (H) Expression of miR-31 and AR in indicated cell lines by qPCR and immunoblot (n=3). (I) VCaP cells treated by vehicle (DMSO) or 5-aza-dC. Left panel and heatmap: DNA methylation levels, right panel: miR-31 levels (n=3). (J) Comparisons of DNA methylation levels at the miR-31 promoter and miR-31 levels between three groups: Gleason score (GS) 6, ≧7, and metastatic cancer(METs). (K) Statistical correlation of the expression of miR-31 and AR in primary PCA samples (n=24) (r=−0.173097, p<0.42). All bar graphs are shown with mean+SEM.

FIG. 2 shows AR and PRC2-mediated repressive histone modification in regulation of miR-31 expression. (A) Expression of miR-31 (left panel) and NDRG1, PSA, and TMPRSS2 (right panel) in LNCaP cells transfected with (as indicated by +, untreated/untransfected cells denoted by −) AR siRNA (siAR) or control siRNA (siCTL), and treated with 1 nM R1881 or vehicle (ethanol), evaluated by qPCR, and AR expression by immunoblot (n=3). (B) Expression of miR-31 and AR in PC3neo cells versus the AR-expressing PC3AR cells, evaluated by qPCR and immunoblot (n=3). (C) Quantitative ChIP analysis with AR, EZH2, and H3K27me3 antibodies at the mIR-31 promoter and regions near miR-31 in LNCaP cells treated with 1 nM R1881 or vehicle (ethanol) (n=3). Red bars represent qPCR regions. (D) Luciferase activity of reporter constructs containing the miR-31 promoter region of −1,000 bp and downstream region+500 bp co-transfected with constructs containing empty vector or AR-CDS with siCTL or siAR in HEK293 cells (n=3, *p<0.01). (E) LNCaP cells in regular medium, miR-31 levels in response to knockdown of AR, EZH2, or both, evaluated by qPCR, and AR expression by immunoblot (n=3). All bar graphs are shown with mean+SEM.

FIG. 3 shows downregulation of AR by miR-31. (A) AR protein level was examined by immunoblot. LNCaP and VCaP cells were transfected with miR-31 or miR-NC (n=3). (B) Expression of PSA and TMPRSS2 evaluated by qPCR (n=3). LNCaP cells transfected with siCTL, siAR, miR-NC, miR-31, and miR-31 with AR-CDS for 48 hours, followed by treatment with 1 nM R1881 or vehicle (ethanol) for 24 hours. (C) Schematic graph illustrating predicted locations of three miR-31 MREs within the transcript of AR variant 1. Numbers in parenthesis correspond to the position in the whole transcript (NM 000044). Perfect matches are shown by a line; G:U pairs by a colon (:). (D) Previously reported mutations are shown in red and the original sequence in bold. Three point mutations, A>G, G>A, and G>T were located within MRE2 and one deletion, ΔG, was located within MRE3. (E) Luciferase activity of LNCaP cells co-transfected with reporter constructs containing WT, mutant (mt), or empty vector (v) and either miR-31 or miR-NC (n=3). (F) AR expression levels in HEK293 cells co-transfected with AR-CDS WT or mutant containing the G>T mutation in MRE2 and either miR-31 or miR-NC, evaluated by qPCR (n=3). (G) AR expression in PC3AR cells transfected with miR-31, miR-NC, inhibitor negative control (IN-NC), or miR-31 inhibitor (IH-miR-31), evaluated by qPCR and immunoblot (n=3). (H) Schematic graph illustrating predicted locations of three miR-31 MREs within the transcript of AR variant 2. Numbers in parenthesis correspond to the position in the whole transcript (NM 001011645). **p<0.01, all bar graphs are shown with mean+SEM.

FIG. 4 shows that genes in cell cycle regulation are direct targets of miR-31. (A) Proliferation assay of LNCaP cells transfected with miR-31 or miR-NC (n=6, * p<0.001). (B) Colony formation analysis of VCaP cells overexpressing miR-31 or vector alone (n=3). (C) cell cycle analysis of LNCaP cells transfected with miR-31 or miR-NC by FACS (n=3). (D) caspase 3/7 activity in LNCaP cells transfected with miR-31 or miR-NC (n=6). (E) Expression of genes involved in cell cycle in LNCaP cells transfected with miR-31 or miR-NC, evaluated by qPCR (n=3). (F) immunoblot of E2F1 with lysates from LNCaP cells transfected with miR-31 or miR-NC (top). Schematic graph illustrates the miR-31 MRE within the 3′UTR of E2F1 (bottom). (G) Luciferase activity of LNCaP cells co-transfected with reporter constructs containing WT or mutant (mt) E2F1 3′UTR or vector alone (v) with either miR-31 or miR-NC (n=3, **p<0.01). (H) Expression levels of indicated proteins from LNCaP cells transfected with miR-31 or miR-NC by immunoblot. (I) Luciferase activity of LNCaP cells transfected with reporter constructs containing 3′UTRs of CDK1, E2F2, EXO1, FOXM1, or MCM2 in conjunction with miR-31 or miR-NC (n=3, *p<0.05, **p<0.01). (J-M) Luciferase activity of LNCaP cells transfected with reporter constructs containing WT or mutant MREs of (J) E2F2 (K) FOXM1, (L) MCM2 and (M) CDK1 in conjunction with miR-31 or miR-NC (n=3, *p<0.05, **p<0.01). All bar graphs are shown with mean±SEM. (N) Putative miR-31 MREs located within the 3′UTR of CDK1, E2F2, EXO1, FOXM1, and MCM2. The numbers in parenthesis correspond to the position in the whole transcript. Perfect matches are shown as a line; G:U pairs by a colon (:).

FIG. 5 shows miR-31 represses PCA growth in vivo. (A-B), luciferase imaging in mice with LNCaP xenografts treated with miR-31 or miR-NC intratumorally. The experiment was terminated after 43 days of initial treatment. (C) Tumors were removed on Day 43 and weighed. (D) Representative immunohistochemistry images of AR (top) and Hematoxylin and eosin staining (H&E) (bottom) in LNCaP xenografts treated with miR-31 or miR-NC. Scale bar: 100 μm. (E) Expression of AR protein levels in LNCaP xenografts treated with miR-31 or miR-NC, evaluated by immunoblot.

FIG. 6 shows (A) the expression of miR-31 attenuated the growth of VCaP xenografts. VCaP cells were transduced by lentiviruses expressing control or miR-31 and implanted subcutaneously to establish VCaP xenografts. Experiments were terminated after 9 weeks. (B) Tumor sizes were measured by caliper every week for 6 weeks and compared. (C) Tumor weights were measured and compared, * p<0.05. (D) Representative immunohistochemistry images of AR in VCaP xenografts from cells transduced with lentiviruses expressing control or miR-31. Hematoxylin and eosin staining (H&E) of the same sections were provided. Scale bar: 100 μm. (E) VCaP xenografts expressing miR-miR-31 showed decreased expression of AR protein levels. AR protein expression was analyzed by immunoblot and β-actin was used as a loading control.

Table 1 shows differentially expressed 105 miRNAs in PCA. FDR adjusted p-value <0.05.

Table 2 shows a list of 25 microRNAs differentially expressed with at least 1.5 fold change in PCA.

Table 3 shows a list of CpG sites and CpG units examined for promoter methylation by MassARRAY EpiTyping.

Table 4 shows a statistical analysis of DNA methylation levels of the miR-31 promoter of 11 matched samples. B=benign tissue, T=PCA. (4.1) Overall difference of DNA methylation levels between benign prostate tissues and PCA. (4.2) Regional difference of DNA methylation levels between benign prostate tissues and PCA. (4.3) List of CpG units showing significant differences between benign prostate tissues and PCA.

Table 5 shows a statistical analysis of DNA methylation levels of the miR-31 promoter in clinical PCA samples. Samples are grouped based on the Gleason scores, three groups are compared: Gleason score=6, Gleason scores ≧7, MET: metastatic PCA. (5.1) Overall DNA methylation levels at the miR-31 promoter region. (5.2) Regional DNA methylation levels of the four divided regions. (5.3) List of CpG units showing significant differences: Gleason score=6 vs. Gleason scores ≧7 and METs. (5.4) List of CpG units showing significant differences: Gleason scores ≧7 vs. METs.

Table 6 shows the top five cellular processes altered by miR-31 in LNCaP cells. Gene Ontology analysis for the whole-genome gene expression profiling in LNCaP cells, miR-31 vs. miR-NC.

Table 7 shows the genomic locations of cytosine nucleotides on chromosome 9 corresponding to the CpG sites and CpG units in Regions 1-4 of the miR-31 promoter referenced in Table 3. The individual strand of chromosomal DNA analyzed is indicated, where nucleotide references are in ascending order on the forward strand and descending order on the reverse strand.

### DETAILED DESCRIPTION OF THE DISCLOSURE

The current disclosure reveals a complex regulatory pattern between miR-31 and AR, indicating that miR-31 plays a key role in prostate cancer development and progression. The current disclosure shows that miR-31 expression is decreased in prostate cancer cells via epigenetic gene silencing processes, including but not limited to DNA promoter methylation. Moreover, DNA methylation of CpG sites in the promoter region of the miR-31 promoter region which is not present in control samples or benign tissue is elevated in cancerous prostate tissue. The current disclosure also shows that AR indirectly represses miR-31 expression by binding to the miR-31 promoter region and modulating methyltransferase activity including, but not limited to increasing EZH2 mediated promoter DNA methylation. Based on these findings, the inventors discovered that the detection of cancer in a subject is enabled by detecting methylated CpG in the promoter region of miR-31. The inventors also discovered that miR-31 modulates oncogenes, transcription factors, and cell-cycle regulatory genes or proteins suppressing their expression or activity. miR-31 has been shown to directly target and destabilize AR mRNA through interaction with the AR mRNA coding sequence showing that miR-31. Accordingly, various diagnostic, prognostic and therapeutic methods with respect to prostate cancer are provided herein.

### TERMINOLOGY

The term “microRNA” or “miRNA” or “miR” refers to small non-coding ribose nucleic acid (RNA) molecules that are capable of regulating the expression of genes through interacting with messenger RNA molecules (mRNA).

The term “miR-31” refers to the small non-coding RNA, microRNA31 (RefSeq NR_{—}029505) which is located between nucleotide 21,512,114 and 21,512,184 MIR31 on chromosome 9 in the intronic region of its host gene MIR31HG (RefSeq NR_{—}027054) located between nucleotide 21,454,267 and 21,559,697 on chromosome 9. The genomic numbering herein is based on GRCh37/hgl9.

The term “AR” refers to the androgen receptor protein or nuclear receptor subfamily 3, group C, member 4 (NR3C4) nuclear receptor.

The term “promoter methylation” or “promoter DNA methylation” refers to the biological process whereby the DNA promoter region of a gene is methylated through the addition of a methyl group to a cytosine, or adenine nucleotide by a methyltransferase. A non-limiting example of promoter methylation includes the addition of methyl groups to cytosine of CpG sites located within the 5′ regulatory region of the MIR31HG gene.

The term “CpG site” refers to locations in a DNA where a cytosine nucleotide occurs next to a guanine nucleotide in the linear sequence of the DNA, i.e., a CG dinucleotide where the cytosine nucleotide is linked to the guanine nucleotide by a phosphodiester bond (hence “CpG”), in contrast to base pairs formed between C and G.

The term “CpG unit” as used herein refers to an analyzed unit containing one or more CpG sites. In quantitative methylation analysis, results are generated for each cleavage product of DNA, and each cleavage product includes either one CpG site or an aggregate of multiple CpG sites in proximity to one another. Thus, the results generated for each unit provides information of the methylation status of all the CpG sites within the unit collectively. Examples of CpG units include those listed in Table 3. For example, in Region 1 of the miR-31 promoter, the first CpG unit, “CpG_{—}1”, contains one CpG site; and the CpG unit “CpG_{—}9.10.11.12” includes four CpG sites, CpG_{—}9, CpG_{—}10, CpG_{—}11, and CpG_{—}12.

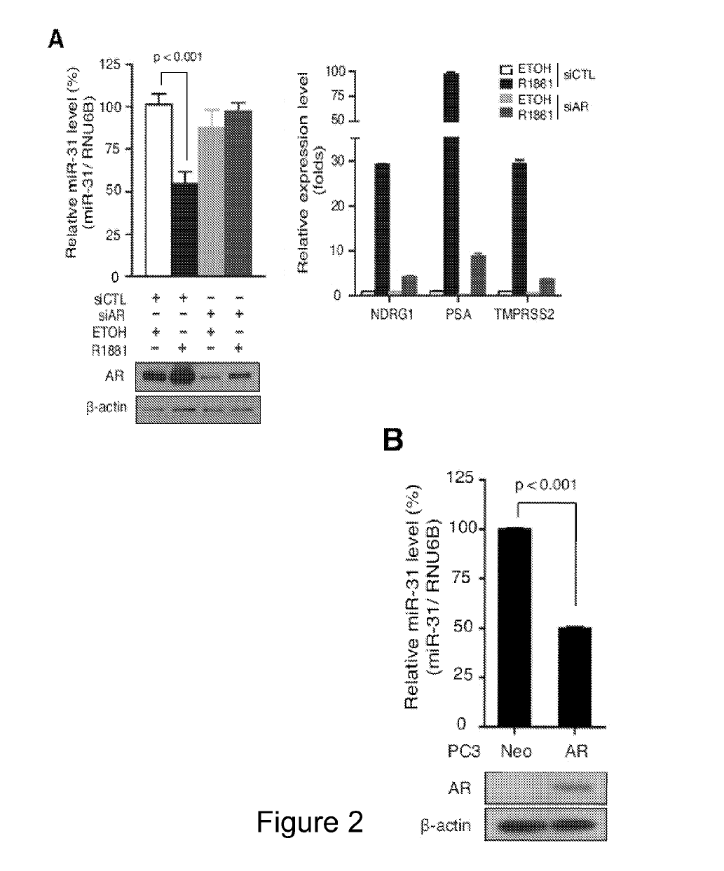

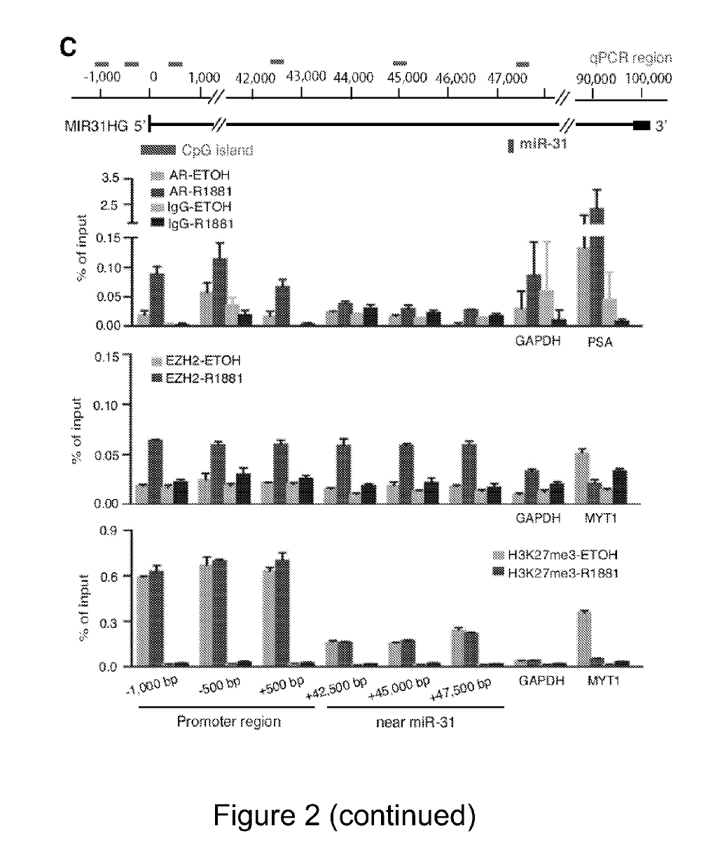

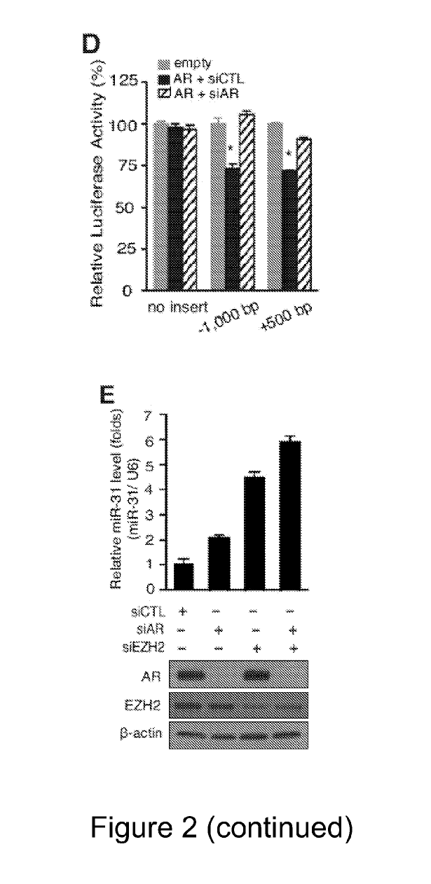

In the present disclosure, the “promoter” region of miR-31 and/or the MIR31HG gene refers to the region of chromosome 9 between nucleotide 21,559,197 and 21,560,697 (i.e., a region of 1500 nucleotides), as shown in FIG. 2c. The miR-31 promoter region can be amplified, for example, using the forward primer CCCCAAGTTATGCACAGGTC [SEQ ID NO:10] and reverse primer CCCTTCAAATCCAGGTGAAA [SEQ ID NO:11]. In another example, the promoter region includes the region downstream from the 5′ end of MIR31HG gene start site amplified by the forward primer TAAAGCAGCTGCCCAATTTT [SEQ ID NO:12] and reverse primer CGAAGTCACAGGTTCGCTCT [SEQ ID NO:13].

The term “CpG island” refers to a DNA region rich in CpGs, wherein the CG content is 50% or more, and typically with an observed-to-expected CpG ratio that is greater than 60%. A non-limiting example is a CpG island located in the miR-31 promoter region, also referred to herein as “miR-31 CpG island”, encompassing nucleotides 21,559,146-21,560,183 of chromosome 9 (i.e., a region of 1,037 nucleotides).

The term “increase” or “greater” means at least more than the relative amount of an entity identified (such as miR-31 expression or miR-31 promoter methylation), measured or analyzed in a control sample. Non-limiting examples, include but are not limited to, 10-20% increase over that of a control sample, or at least 20%, 30%, 40%, 50%, 60%, 70%, 80%, 90%, 100%, 200% or greater increase over that of a control sample, or at least 1 fold, 1.5 fold, 2 fold, 3 fold or greater, increase relative to the entity being analyzing in the control sample.

The term “decrease” or “reduction” means at least lesser than the relative amount of an entity identified, measured or analyzed in a control sample. Non-limiting examples, include but are not limited to, 10-20% decrease over that of a control sample, or at least 20%, 30%, 40%, 50%, 60%, 70%, 80%, 90%, 100%, 200% or greater decrease over that of a control sample, or at least 1 fold, 1.5 fold, 2 fold, 3 fold or greater, decrease relative to the entity being analyzing in the control sample.

An “increase in miR-31 expression” as used in the current disclosure shall mean an increase in the amount of miR-31.

The phrase “modulating the activity” or “modulating the level” is employed herein to refer to increasing the level or activity; or decreasing the level or activity of an entity including but not limited to, a gene, peptide or molecule within a cell. Non-limiting examples of the activity or level of a molecule of the current disclosure includes the amount of microRNA present in a cell, or sample. In an aspect of the current disclosure, the amount of microRNA present is the level of miR-31 present in a cell or sample. Yet another example of the activity or level as utilized in the current disclosure is the amount of androgen receptor (AR) protein in a cell, or sample; or the ability miR-31 or AR to function or bind effectors thereof

The phrase “miR-31 activity” or “miR-31 function” refers to the ability of miR-31 to bind effectors, respond to changes in cellular conditions, or regulate cellular homeostasis. A non-limiting example of miR-31 activity is the ability of miR-31 to bind AR, or other cell cycle regulators.

The phrase “AR activity” or “AR function” refers to the ability of a the androgen receptor protein to bind androgen, interact with effectors thereof, respond changes in cellular conditions, or regulate cellular homeostasis. A non-limiting example of AR activity is the ability of AR to bind androgens, for example, dehydroepiandrosterone (DHEA) and androsteindione; chaperonin proteins, including but not limited to, HSP90; testosterone, including but not limited to dihydrotestosterone (DHT); other cell cycle regulators, including but not limited to, kinases Ack1 and SRC; or inhibiting the ability of AR to mediate transcription or AR target genes, including but not limited to TMPRSS-ETS.

The term “agent” is employed herein to refer to any kind of compound, molecule or ion and any combination thereof. In one embodiment of the disclosure the agent is a small molecule. In another embodiment of the disclosure, the agent is a biological molecule, including, but not limited to, a protein or a peptide or a nucleic acid, or an ion. In another embodiment, the nucleic acid is an interfering RNA. In yet another embodiment, the agent is an antibody or fragment thereof.

The phrase “effector” or “effectors” refers to any small molecule, protein, ligand, or complex thereof that binds to, or interacts with miR-31 or AR either directly or indirectly. The result of this interaction may modulate a biological activity, including but not limited to, cell cycle progression, proliferation, transcription, DNA repair, DNA replication, protein-protein interaction, protein-DNA interaction, RNA-RNA interaction or cellular signaling. Non-limiting examples of AR effectors include androgen, AKT1, Beta-catentin, cyclin D1, cyclin dependent kinase (CDK) 7, epidermal growth factor receptor (EGFR), Foxol, GAPDH, HDAC1, HSP90, PTEN, SMAD3, Src, STAT3, testosterone, Ack1 and SRC.

The term “interfering RNA” is employed herein to refer to small interfering RNAs (siRNAs), short hairpin RNAs (shRNAs), microRNAs (miRNAs), antisense oligonucleotides, ribozymes, or any RNA-based molecule that interferes with the expression of a protein from its corresponding gene or modulate the activity of the protein.

In the context of this disclosure, the term “small molecule” refers to small organic compounds, including but not limited to, heterocycles, peptides, saccharides, steroids, antibodies and the like. The small molecule modulators can have a molecular weight of less than about 1500 Daltons, 1200 Daltons, 1000 Daltons, or 800 Daltons. In some embodiments, a small molecule modulator is less than 500 Daltons. The small molecules can be modified to enhance efficacy, stability, pharmaceutical compatibility, and the like. Candidate modulator compounds from libraries of synthetic or natural compounds can be screened. Synthetic compound libraries are commercially available from a number of companies including Maybridge Chemical Co. (Trevillet, Cornwall, UK), Comgenex (Princeton, N. J.), Brandon Associates (Merrimack, N.H.), Microsource (New Milford, Conn.), and ChemBridge (San Diego, Calif.). Combinatorial libraries are available or can be prepared according to known synthetic techniques. Alternatively, libraries of natural compounds in the form of bacterial, fungal, plant and animal extracts are available from e.g., Pan Laboratories (Bothell, Wash.) or MycoSearch (NC), or are readily producible by methods well known in the art. Additionally, natural and synthetically produced libraries and compounds may be further modified through conventional chemical and biochemical techniques.

The term “peptide” refers to a linear series of amino acid residues linked to one another by peptide bonds between the alpha-amino and carboxy groups of adjacent amino acid residues.

The term “synthetic peptide” is intended to refer to a chemically derived chain of amino acid residues linked together by peptide bonds. The term “synthetic peptide” is also intended to refer to recombinantly produced peptides in accordance with the present disclosure.

The phrase “subject in need thereof” as used herein refers to any mammalian subject in need of treatment, or requiring preventative therapy to prevent a condition resulting from lower or higher than normal levels of miR-31 expression or activity or AR expression or activity in the organism. The methods of the current disclosure can be practiced on any mammalian subject that has a risk of developing cancer. Particularly, the methods described herein are most useful when practiced on humans.

The term “effective amount” is employed herein to refer to the amount of an agent that is effective in modulating miR-31 levels, miR-31 activity or AR levels, activity or function in a subject or cell.

A “biological sample,” “sample” or “samples” to be used in the disclosure can be obtained in any manner known to a skilled artisan. Samples can be derived from any part of the subject, including whole blood, urine, tissue, lymph node or a combination thereof. More specifically a sample includes tissue samples, for example a tissue biopsy sample from a tissue having or suspected of having cancer, or a tissue sample from a portion of a surgically removed tumor. Samples of the current disclosure may be processed according to any of the methods described herein. Conversely, a “control sample” is a sample known not to possess cancerous cells, and not to exhibit increased miR-31 levels or activity. Non-limiting examples of control samples for use in the current disclosure include, non-cancerous tissue extracts, isolated cells known to have normal miR-31 levels and normal AR activity, obtained from the subject under examination or other healthy individuals. In one aspect, the control sample of the present disclosure is benign prostate tissue. In one embodiment of the current disclosure, the amount of microRNA in a sample is compared to either a standard amount of the microRNA present in a normal cell or a non-cancerous cell, or to the amount of microRNA in a control sample. The comparison can be done by any method known to a skilled artisan.

A wide variety of chemotherapeutic agents may be used in accordance with the disclosed methods. A “chemotherapeutic agent” or “chemotherapeutic treatment” is used to connote a compound or composition that is administered in the treatment of cancer. These agents or drugs are categorized by their mode of activity within a cell, for example, whether and at what stage they affect the cell cycle. Alternatively, an agent or treatment may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas. Non-limiting examples of chemotherapeutic agents or chemotherapeutic treatments include methotrexate, fluorouracil (5-FU), docataxel, doxorubicin, cisplatin, ifosfamide and ralitrexed.

An “AR targeting therapeutic agent” is any composition, small molecule, antibody, nucleic acidic, antisense nucleic acid molecule, shRNA, siRNA or combination thereof that modulates androgen receptor function, activity or expression. Non-limiting examples of AR targeting therapeutic agents, include but are not limited to, GnRH agonists, antiandrogens, including but not limited to, MDV-3100, BMS-641988, dutasteride, finasteride, abirateroneinhibitors of CYP17, inhibitors or 5α-reductase, inhibitors of HSP90, HDAC inhibitors, including but not limited to, suberoylanilide hydroxamic acid (SAHA), trichostatin A, depsipeptide and tyrosine kinase inhibitors for example, dasatinib, erlotinib, lapatinib, trastuzamab or pertuzumab.

The term “binding”, “to bind”, “binds”, “bound” or any derivation thereof refers to any stable, rather than transient, chemical bond between two or more molecules, including, but not limited to, covalent bonding, ionic bonding, and hydrogen bonding. Thus, this term also encompasses hybridization between two nucleic acid molecules among other types of chemical bonding between two or more molecules.

“LNCaP” refers to a human cell line established from metastatic prostate adenocarincoma. The cell line is sensitive to androgen as androgen receptor proteins are present in abundance (see Horoszewicz, J S., et al., Cancer Res. (1983) 43(4): 1809-1818).

“VCaP” as referred to in the current disclosure means Vertebral-Cancer of the Prostate cell line. These cells were established from metastatic prostate cancer tissue extracted from a patient with hormone refractory prostate cancer (see Korenchuck, S., et al. In Vivo (2001) 15(2):163-8).

Herein, the term “substantially identical” when used in reference to nucleotide sequences, refers to a nucleotide sequence having an identity of at least 60%, 70%, 80%, 90%, 95%, 98% or greater to a specified nucleotide sequence.

### Diagnostic and Prognostic Methods

In one aspect, the present disclosure provides a method of diagnosing prostate cancer in a subject based on detecting altered miR-31 promoter methylation.

A biological sample is obtained from the subject in question. The biological sample that can be used in accordance with the present disclosure may be collected by a variety of means. Non-limiting examples include, by surgery, by paracentesis needle for tissue collection, or by collection of body fluid, secretion from a gland, blood extract or urine. In some embodiments, the sample obtained from a subject is used directly without any preliminary treatments or processing, such as fractionation or DNA extraction. In other embodiments, the sample is processed such that DNA can be extracted or enriched from the sample before detecting expression levels or DNA methylation. Methods of extracting DNA from biological sample are well known in the art, and may be performed using, for example, phenol/chloroform, ethanol, or commercially available DNA extraction reagents.

After a suitable biological sample is obtained, the level of miR-31 promoter methylation in the sample can be determined using various techniques. In certain embodiments of the current disclosure miR-31 promoter methylation is measured by a process selected from methylation-specific polymerase chain reaction, single-molecule, real-time sequencing, bisulfite DNA sequencing, HPLC, mass spectrometry, microarray, methylation-specific PCR, bisulfite sequencing, pyro-sequencing, combined bisulfate restriction analysis (COBRA), or MethyLight or methylated DNA immunoprecipitation.

Methylation-specific PCR utilizes the difference in sensitivity to the cytosine-to-uracil conversion in the presence or absence of methylation, generating primers specific to methylated allele and unmethylated allele, amplifying and detecting them by PCR for detection. The obtained PCR product is subjected to electrophoresis on an agarose gel, stained with ethidium bromide, then the presence of methylation is determined by the presence of bands (see, Eads, C A., et al., Methods Mol Biol. (2002) 200:71-85).

Bisulfite sequencing utilizes bisulfite-treated DNAs to amplify methylated and unmethylated DNA sequences at the same time, and the obtained PCR products are then cloned into cloning vectors known to those skilled in the art, and then the presence or absence of methylation is detected by sequencing. (see Warnecke P M., et al Methods. (2002) 27(2): 101-7).

Pyro-sequencing uses bisulfite-treated DNAs to amplify methylated and unmethylated alleles at the same time, then analyzes obtained PCR products by pyro-sequencing. The ratio of methylation is detected as the polymorphism of cytosine and thymine, calculated as [fluorescent intensity of cytosine/the sum of fluorescent intensities of cytosine and thymine]. It is useful as a quantitative and high-throughput methylation analysis (see Tost J. et al., Nat Protoc. (2007) 2(9):2265-75).

COBRA method uses bisulfite-treated DNAs to amplify methylated and unmethylated alleles at the same time, and obtained PCR products are digested with restricted enzymes, then subjected to electrophoresis on an agarose gel, stained with ethidium bromide, and the presence or absence of methylation is determined according to the presence or absence of the bands cleaved by restriction enzymes (see Eads, C A., et al., Methods Mol Biol. (2002) 200:71-85).

MethyLight method detects methylated alleles using methylation-specific PCR combined with TaqMan PCR. It is capable of highly sensitive detection of methylation, and is useful in detection of methylation from small amount of specimen (see Trinh, B N., et al. Methods. (2001) 25(4):456-62).

When using methylation-specific PCR, bisulfite sequencing, pyro-sequencing, COBRA and MethyLight, an unmethylated cytosine is specifically converted to a uracil whereas a methylated cytosine is not converted to an uracil due to bisulfite treatment.

In a further non-limiting example, matrix-assisted laser desorption ionization/time-of-flight (MALDI-TOF) mass spectrometry is conducted on bisulfite treated DNA. Wherein, genomic DNA is isolated using standard techniques, including but not limited to phenol-chloroform and then purified by ethanol precipitation. The isolated DNA is then treated with bisulfite, whereby the cytosine nucleotides that are not present as methylated cytosine molecules are replaced by uracil. Next, in order to obtain a sufficient amount of DNA to analyze, PCR can be performed that will result in DNA polymerase converting uracil to thymine and the methylated cytosine to unmethylated cytosine

As demonstrated herein, significant alterations in methylation across the entire CpG island of the miR-31 promoter have been observed in prostate cancer samples relative to control. Hence, according to this disclosure, analysis of methylation can be directed to the miR-31 promoter region, especially the miR-31 CpG island in the miR-31 promoter region, or a portion or fragment within the miR-31 island.

In an embodiment of the current disclosure, the entire miR-31 CpG island (e.g., a region consisting of or encompassing the CpG island) is amplified for methylation analysis. In another embodiment, one or more fragments of the miR-31 CpG island are analyzed. For purposes of methylation analysis, a “fragment” or “portion” refers to a fragment of between 100-500 nucleotides in length, between 150-400 nucleotides in length, or between 200-300 nucleotides in length. In specific embodiments, the region analyzed is about 200 nucleotides in length, 250 nucleotides in length, 300 nucleotides in length, 350 nucleotides in length, or a length between any of the above listed values. In some embodiments, the fragment of the miR-31 island being analyzed contains at least one, i.e., one or more, of the CpG units listed in Table 3. In some embodiments, the fragment of the miR-31 island being analyzed contains at least 2, 3, 4, 5, 6, 7, 8, 9, 10, 11, 12, 13, 14, 15 or more of the CpG units listed in Table 3.

Non-limiting examples of fragments suitable for miR-31 promoter methylation analysis include Region 1, Region 2, Region 3, and Region 4, as further described and illustrated in the examples herein below. Region 1 refers to the region in the miR-31 promoter between nucleotides 21,559,054 and nucleotide 21,559,490 of chromosome 9; Region 2 refers to the region in the miR-31 promoter between nucleotides 21,559,412 and 21,559,763 of chromosome 9; Region 3 refers to the region in the miR-31 promoter between nucleotides 21,559,742 and 21,560,029 of chromosome 9; and Region 4 refers to the region in the miR-31 promoter between nucleotides 21,559,977 and 21,560,161 of chromosome 9.

These regions can be amplified by using appropriate primer pairs, e.g., for Region 1: a forward primer encompassing the nucleotide sequence TTTTTTTTAAGAAGGGAAAGTTTAG [SEQ ID NO: 2] and a reverse primer encompassing CAAATAAACTAAAAAAACCTTAATCCC [SEQ ID NO: 3]; Region 2: a forward primer encompassing the sequence TTTTTAGGAGGAGTTTGGTGAGTAG [SEQ ID NO: 4] and a reverse primer encompassing the sequence AACCTCCTCAACTCCTTAAAA [SEQ ID NO: 5]; Region 3: a forward primer encompassing the sequence of GTTGTAGTGGAGAAATTTGGGTTT [SEQ ID NO: 6] and a reverse primer encompassing the sequence of CACCAAACTCCTCCTAAAAA [SEQ ID NO: 7]; and Region 4: a forward primer encompassing the sequence of TGGTTTTTGTAGGTGGATTTTTTT [SEQ ID NO: 8] and a reverse primer encompassing the sequence of TACTAAACCTCCTCCCTTAAACCC [SEQ ID NO: 9].

One of ordinary skill in the art can design primers to amplify and/or clone a desired region of DNA by conventional methods, including but not limiting to adding any number of nucleotide base pairs selected from the group consisting of, A (Adenine), T (Thymine), C (Cytosine) and G (Guanine) to the 5′ or 3′ end of the desired DNA sequence (e.g., a sequence containing a restriction enzyme cleavage site or a sequence recognized by a DNA polymerase for initiation of transcription) to enable convenient cloning and/or efficient transcription of the target sequence being amplified or cloned. A non-limiting example includes adding nucleotides the DNA sequence of the T7 promoter (CAGTAATACGACTCACTATAGGGAGAAGGCT [SEQ ID NO:14].

In some embodiments, promoter methylation is analyzed for each of Regions 1-4 alone or in combination. For example, methylation of one of Regions 1-4 (i.e., Region 1, 2, 3 or 4) is being analyzed, the measurement of this region alone is used as basis for diagnosis. As another example, multiple regions (i.e., two, three or all four of Regions 1-4) are being analyzed, and the collective measurements of methylation of these regions are used as basis for diagnosis. Non-limiting examples include, the amplification and subsequent analysis of Region 1, 2 and 3, or Region 1, 2 and 4, or Region 1, 3 and 4, or Region 2, 3 and 4, or Region 1 and 2, or Region 1 and 3, or Region 1 and 4, or Region 2 and 3, or Region 2 and 4, or Region 3 and 4.

In another embodiment, methylation is detected for certain CpG units within the miR-31 promoter region. Nucleic acid regions encompassing specific, desirable CpG units can be amplified for analysis. In specific embodiments of the current disclosure, the CpG units analyzed include one or more of CpG_{—}9.10.11.12, CpG_{—}19.20.21, and CpG_{—}22.23 within Region 1.

In specific embodiments of the current disclosure, one or more CpG unit-containing fragments within the miR-31 island are amplified for methylation analysis of bisulfate treated DNA. After a bisulfate-treated CpG unit-containing nucleic acid region has been PCR amplified, in vitro RNA transcription is performed on the reverse strand, followed by a base specific cleavage. The cleavage product results in a distinct signal pattern from the methylated and non-methylated template DNA that is automatically and quantitatively measured with the MassARRAY system. The resulting cleavage pattern, which connotes the amount of methylation present in a sample depends on the presence of methylated cytosine in the original genomic DNA. The methylation content ratios can be further analyzed using standard statistical methods or software programs, including but not limited to, EpiTYPER software v1.0 (Sequenom).

In one embodiment, the miR-31 promoter methylation measured from the subject in question is compared to a control value in order to determine whether or not prostate cancer exists in the subject.

An alteration evidenced by an increase in promoter methylation relative to a control value indicates that the subject has prostate cancer. Alternatively, when the level of miR-31 promoter methylation is decreased or equal to a control value, it can be determined that said subject does not have prostate cancer. A control value can be a pre-determine value or can be determined from a control sample side by side with the sample obtained from the subject in question.

In yet another embodiment, the control value is established from a control sample obtained from benign tissue, including but not limited to benign prostate tissue. That is, the level of miR-31 promoter methylation in a test sample is compared to that of a sample obtained from benign tissue, including but not limited to benign prostate tissue. If the amount of microRNA DNA methylation in the test sample is greater than the amount of microRNA DNA methylation in the control sample, then the subject is diagnosed as having prostate cancer.

According to this disclosure, the methylation levels of the CpG units within a region in a test sample are analyzed and are compared to the methylation levels of the corresponding CpG units from a control (e.g., a sample from benign tissue), and the difference in methylation between the test sample and the control can be determined. In one approach, an average methylation level is calculated based on the levels of all of the CpG units within the region examined, and this average is compared to an average control (e.g., calculated based on the levels of all the CpG units within the region in a control sample). In another approach, the methylation level of each CpG unit of a test sample is compared to a control level (e.g., the methylation level of the same CpG unit in a control sample), and alteration is determined for each CpG unit within the region being examined, and an average alteration is calculated based on all the alterations determined for all the CpG units in the region. Alterations evidenced by a significant increase in methylation of a test sample relative to a control indicate that the test subject has prostate cancer.

Non-limiting examples of a significant increase in promoter DNA methylation, include but are not limited to, 2%, 3%, 4%, 5%, 6%, 7%, 8%, 9%, 10%, 15%, 20%, 30%, 40%, 50% or more, over that of a control. Non-limiting examples of a decrease in promoter DNA methylation, include but are not limited to, 5%, 10%, 15%, 20%, 25%, 30% or more over a control.

In yet another embodiment of the current disclosure, the level of miR-31 promoter methylation is used as a basis to determine the severity of prostate cancer in the subject. According to the current disclosure, the extent of the increase in miR-31 promoter methylation relative to the control correlates with the stage of the cancer; that is, the more elevated miR-31 promoter methylation is, the more advanced the prostate cancer is, and hence the expected response of treating the subject with a chemotherapeutic agent is lower, and/or the expected survival of the subject is lower. In a non-limiting example the association between Gleason score and promoter methylation was determined, whereby the average of tumor grade % (outcome variable) was calculated for subject and a standard descriptive summary was then performed for this grade % average stratified by three categories (Gleason score=6, ≧7, and metastatic PCA). Kruskal-Wallis nonparametric test or Wilcoxon rank-sum test was used to evaluate the difference among three or between two of the three categories, respectively.

The disclosure further provides a method of diagnosing prostate cancer in a subject by determining the level of expression of miR-31. A biological sample is obtained from the subject in question. The biological sample that can be used in accordance with the present disclosure may be collected by any means. Non-limiting examples include, by surgery, by paracentesis needle for tissue collection, or by collection of body fluid, secretion from a gland, blood extract or urine. In some embodiments, the sample obtained from a subject is used directly without any preliminary treatments or processing, such as fractionation or DNA extraction. In other embodiments, the sample is processed such that DNA can be extracted or enriched from the sample before detecting expression levels or DNA methylation. Methods of extracting DNA from biological sample are well known in the art, and may be performed using, for example, phenol/chloroform, ethanol, or commercially available DNA extraction reagents. The level of miR-31 expression is then measured in the sample, wherein the level of miR-31 expression indicates the presence or absence of prostate cancer in the subject. Measuring the amount of microRNA can be performed in any manner known by one skilled in the art of measuring the quantity of RNA within a sample. A non-limiting example of a method for quantifying microRNA is quantitative reverse transcriptase polymerase chain reaction (qPCR). In an aspect of the current disclosure, microRNA expression profiling can be performed using microarray technology (see e.g. Liu, C G., et al., Nature Protocols (2008). (3):563-578; Kerscher, A., et al., Nature Methods (2004). (1):106-107). In one aspect, real-time reverse transcriptase PCR is conducted, wherein a stem-loop primer is hybridized to the microRNA molecule to be detected, then reverse transcribed with reverse transcriptase enzymes, and then quantified using real-time PCR (see e.g. Chen, C., et al. Nucleic Acids Res. (2005). 33(20): e179). Yet, another example of a method of quantifying microRNA is as follows: hybridizing at least a portion of the microRNA with a second nucleic acid or oligonucleotide, and reacting the hybridized microRNA with a fluorescent reagent, wherein the hybridized microRNA emits a fluorescent light. Another method of quantifying the amount of microRNA in a sample is by hybridizing at least a portion the microRNA to a radio-labeled complementary nucleic acid. In instances when a nucleic acid capable of hybridizing to the microRNA is used in the measuring step, the nucleic acid is at least 5 nucleotides, at least 10 nucleotides, at least 15 nucleotides, at least 20 nucleotides, at least 25 nucleotides, at least 30 nucleotides or at least 40 nucleotides. Wherein the binding of the nucleic acid to the target microRNA is diagnostic for cancer.

The level of miR-31 expression in the sample can be compared to that of a control sample. A control sample can be obtained from benign tissue, including but not limited to benign prostate tissue, from the test subject or other normal healthy individuals. In a non-limiting example of the current disclosure, if the amount of microRNA in the sample is lower than the amount of microRNA in the control sample, then the subject is diagnosed as having cancer. According to this disclosure, the decrease of miR-31 expression relative to control should be at least 10%, 20%, 30%, 40%, 50%, 60%, 70%, 80%, 90%, 100%, 200% or greater, in order to make a diagnosis.

In yet another embodiment of the current disclosure, the level of miR-31 expression forms a basis for determining the severity of prostate cancer in the subject. More specifically, the level of miR-31 expression is inversely correlated with the stage of the prostate cancer; that is, the lower the miR-31 expression, the more advanced the prostate cancer. Additionally, the subject's expected response to therapeutic treatment, and/or the expected survival of the subject can also be determined based on the level of miR-31 expression. For example, if the expression of the microRNA in the sample is lower than the expression of the microRNA in a control sample, this result indicates a poor prognosis for a subject to positively respond to chemotherapeutic treatment.

A non-limiting example of the above embodiments encompasses when a reduction in the level of miR-31 expression is present in a sample or miR-31 DNA methylation is elevated, then the subject is less likely to positively respond to a chemotherapeutic treatment. Another non-limiting example is that if the amount of miR-31 expression is elevated or miR-31 DNA methylation is reduced in a sample, when compared to the control sample or standard then the subject is likely to positively respond to a treatment with a chemotherapeutic agent.

Another non-limiting example is that if the amount of miR-31 expression is reduced or miR-31 DNA methylation is elevated, then the subject is less likely to survive the cancer as compared to a subject with cancer who has normal levels of miR-31 expression or miR-31 promoter DNA methylation, or to the expected survival of an average subject having cancer. The converse is also true. Thus, if the amount of miR-31 expression or miR-31 promoter DNA methylation is normal in sample from a subject diagnosed with cancer, then the subject has a positive prognosis for surviving the cancer, the subject is more likely to survive the cancer or the subject is more likely to survive the cancer longer than an average subject inflicted with the cancer extend life as compared to a subject that over-expresses miR-31 or has increased miR-31 promoter DNA methylation.

### Therapeutic Methods

The disclosure further provides a method of treating a prostate cancer in a subject, by administering to the subject an effective amount of an agent that modulates the activity of miR-31. In one embodiment of the current disclosure said agent is a nucleic acid, a small molecule, an antibody or a peptide, any of which directly or indirectly modulates the expression of miR-31, modulates miR-31 promoter methylation, or modulates the interaction between miR-31 and a regulator of cell cycle progression. Non-limiting examples of modulating cell cycle progression includes the inhibition of cell proliferation, colony formation and cell cycle arrest via modulation of transcription factors, including but not limited to, the repression of E2F1, E2F2, EXO1, FOXM1, and MCM2; or through the modulation of cyclin dependent kinases, including but not limited to, CDK1, which indirectly contributes to AR downregulation. In yet another embodiment a second therapeutic agent is provided to the subject including administration of a chemotherapeutic agent, radiation or an AR targeting therapeutic agent.

The disclosure further provides an isolated nucleic acid that modulates the activity of the androgen receptor in a cell. In a further embodiment, the isolated nucleic acid is miR-31 [SEQ ID NO:1] or an isolated nucleic acid that is substantially identical to the nucleotide sequence of miR-31 [SEQ ID NO:1]. Non-limiting examples of said substantially identical nucleic acids of the current disclosure include, UCGAUACGGUCGUAGAACGGA [SEQ ID NO:1]; UCGN₃CNGUCNUAGAACNGN [SEQ ID NO:15]; UCNANNCGGUNNCGNNGAACGGA [SEQ ID NO:16]; N₆CGGUCN₈ANAACGGN [SEQ ID NO:17].

Where, N can be any nucleotide independently selected from:

### A=Adenine

### C=Cytosine

### G=Guanine

### U=Uracil

An isolated nucleic acid molecule or agent of the current disclosure may be administered within a pharmaceutically-acceptable diluents, carrier, or excipient, in unit dosage form. As described herein, if desired, treatment with an isolated nucleic acid molecule of the current disclosure may be combined with therapies such as, for example, radiotherapy, surgery, chemotherapy or an AR targeting therapy for the treatment of prostate cancer.

The dosage of an agent that is administered to a subject in need thereof may vary, depending on the reason for use and the individual subject. The dosage may be adjusted based on the subject's weight, the age and health of the subject, and tolerance for the agent.

The amount of agent (therapeutic) to be used depends on many factors. Dosages may include about 2 mg/kg of bodyweight/day, about 5 mg/kg of bodyweight/day, about 10 mg/kg of bodyweight/day, about 15 mg/kg of bodyweight/day, about 20 mg/kg of bodyweight/day, about 25 mg/kg of bodyweight/day, about 30 mg/kg of bodyweight/day, about 40 mg/kg of bodyweight/day, about 50 mg/kg of bodyweight/day, about 60 mg/kg of bodyweight/day, about 70 mg/kg of bodyweight/day, about 80 mg/kg of bodyweight/day, about 90 mg/kg of bodyweight/day, about 100 mg/kg of bodyweight/day, about 125 mg/kg of bodyweight/day, about 150 mg/kg of bodyweight/day, about 175 mg/kg of bodyweight/day, about 200 mg/kg of bodyweight/day, about 250 mg/kg of bodyweight/day, about 300 mg/kg of bodyweight/day, about 350 mg/kg of bodyweight/day, about 400 mg/kg of bodyweight/day, about 500 mg/kg of bodyweight/day, about 600 mg/kg of bodyweight/day, about 700 mg/kg of bodyweight/day, about 800 mg/kg of bodyweight/day, and about 900 mg/kg of bodyweight/day. Routine experimentation may be used to determine the appropriate value for each patient by monitoring the compound's effect on KCNQ channel activity, or the disease pathology, which can be frequently and easily monitored. The agent can be administered once or multiple times per day. The frequency of administration may vary from a single dose per day to multiple doses per day. Routes of administration include oral, intravenous and intraperitoneal, but other forms of administration may be chosen as well.

The effective amount of an agent according to the present disclosure may be administered along any of the routes commonly known in the art. This includes, for example, (1) oral administration; (2) parenteral administration, for example, by subcutaneous, intramuscular or intravenous injection; (3) topical administration; or (4) intravaginal or intrarectal administration; (5) sublingual or buccal administration; (6) ocular administration; (7) transdermal administration; (8) nasal administration; and (9) administration directly to the organ or cells in need thereof.

The effective amount of an agent according to the present disclosure may be formulated together with one or more pharmaceutically acceptable excipients. The active ingredient and excipient(s) may be formulated into compositions and dosage forms according to methods known in the art. These compositions and dosage forms may be specially formulated for administration in solid or liquid form, including those adapted for the following: (1) oral administration, for example, tablets, capsules, powders, granules, pastes for application to the tongue, aqueous or non-aqueous solutions or suspensions, drenches, or syrups; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of the subject with toxicity, irritation, allergic response, or other problems or complications, commensurate with a reasonable benefit/risk ratio.

The phrase “pharmaceutically-acceptable excipient” as used herein refers to a pharmaceutically-acceptable material, composition or vehicle, such as a liquid or solid filler, diluent, carrier, manufacturing aid (e.g., lubricant, talc magnesium, calcium or zinc stearate, or stearic acid), solvent or encapsulating material, involved in carrying or transporting the therapeutic compound for administration to the subject. Each excipient should be “acceptable” in the sense of being compatible with the other ingredients of the formulation and not injurious to the subject. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19^{th} Ed. Mack Publishing Company, Easton, Pa., (1995).

Excipients are added to the agent for a variety of purposes. Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel®), microfinc cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical agents that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the subject's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid agent and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

In liquid pharmaceutical compositions of the present disclosure, the agent and any other solid excipients are dissolved or suspended in a liquid carrier such as water, water-for-injection, vegetable oil, alcohol, polyethylene glycol, propylene glycol or glycerin. Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol. Liquid pharmaceutical compositions of the present disclosure may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste. Flavoring agents and flavor enhancers may make the dosage form more palatable to the patient. Common flavoring agents and flavor enhancers for pharmaceutical products that may be included in the composition of the present disclosure include maltol, vanillin, ethyl vanillin, menthol, citric acid, fumaric acid, ethyl maltol and tartaric acid.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present disclosure, a liquid composition may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

Solid and liquid compositions may also be dyed using any pharmaceutically acceptable colorant to improve their appearance and/or facilitate patient identification of the product and unit dosage level.

The dosage form of the present disclosure may be a capsule containing the composition, for example, a powdered or granulated solid composition of the disclosure, within either a hard or soft shell. The shell may be made from gelatin and optionally contain a plasticizer such as glycerin and sorbitol, and an opacifying agent or colorant.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant. A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling may include any of the aforementioned blends and granulates that were described with reference to tableting; however, they are not subjected to a final tableting step.

In the context of the present disclosure, the effective amount of the agent modulating the activity of miR-31 or AR activity may be administered alone or in combination with one or more additional therapeutic agents (“second therapeutic agent”), regardless of the disease that said second therapeutic entity is administered to treat. In a combination therapy, the effective amount of the agent modulating miR-31 activity or AR activity may be administered before, during, or after commencing therapy with a second therapeutic agent, as well as any combination thereof, i.e., before and during, before and after, during and after, or before, during and after commencing the additional therapy. For clarity, an agent of the present disclosure may be administered in an effective amount in response a prior treatment that brings about the need to modulate miR-31 or AR expression or activity. The current disclosure shows that AR indirectly represses miR-31 expression by binding to the miR-31 promoter region and modulating methyltransferase activity including, but not limited to increasing EZH2 mediated promoter DNA methylation.

It is to be understood that this invention is not limited to particular methods, and experimental conditions described, as such methods and conditions may vary. It is also to be understood that the terminology used herein is for the purpose of describing particular embodiments only, and is not intended to be limiting.

### EXAMPLES

The following examples further illustrate the disclosure, but should not be construed to limit the scope of the disclosure in any way.

### Example 1

### MiR-31 Expression is Suppressed in PCA

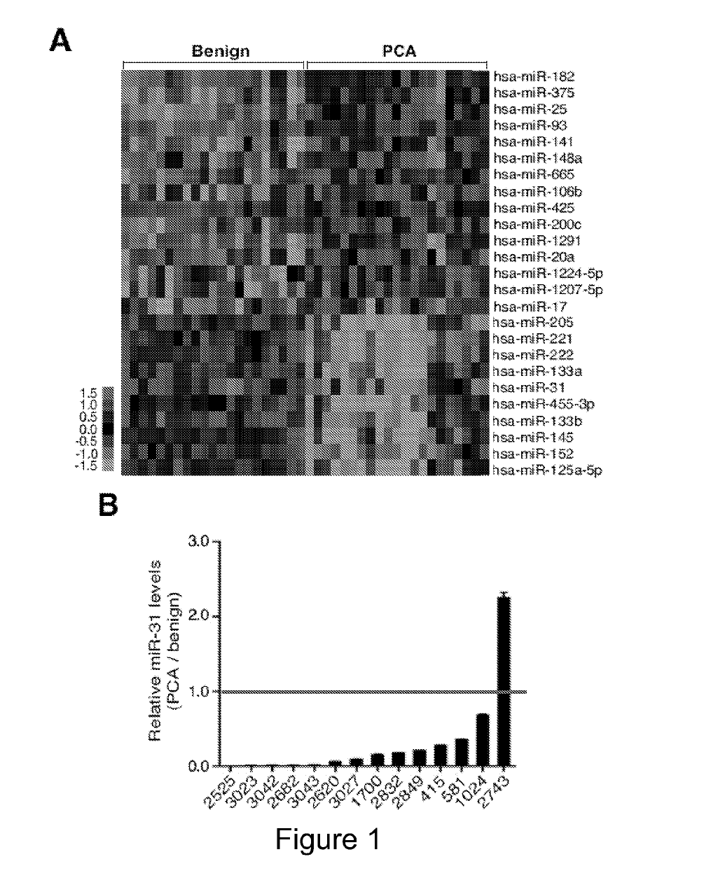

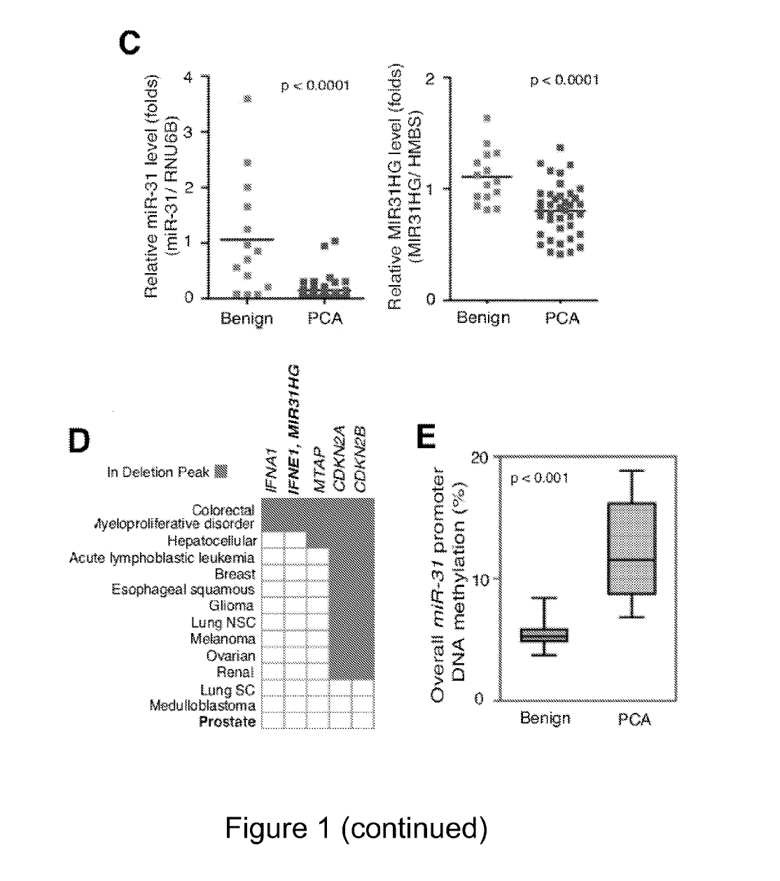

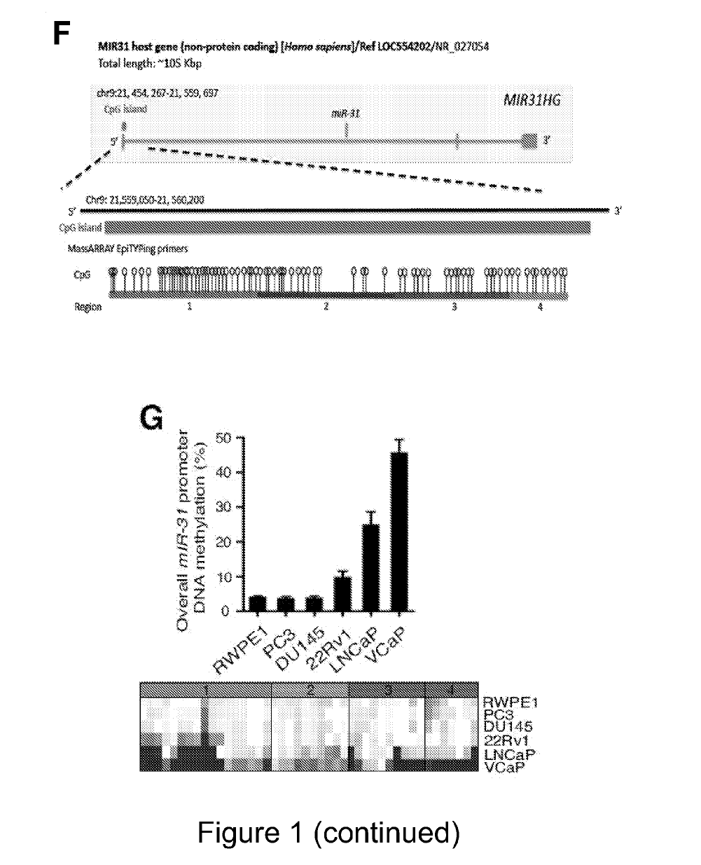

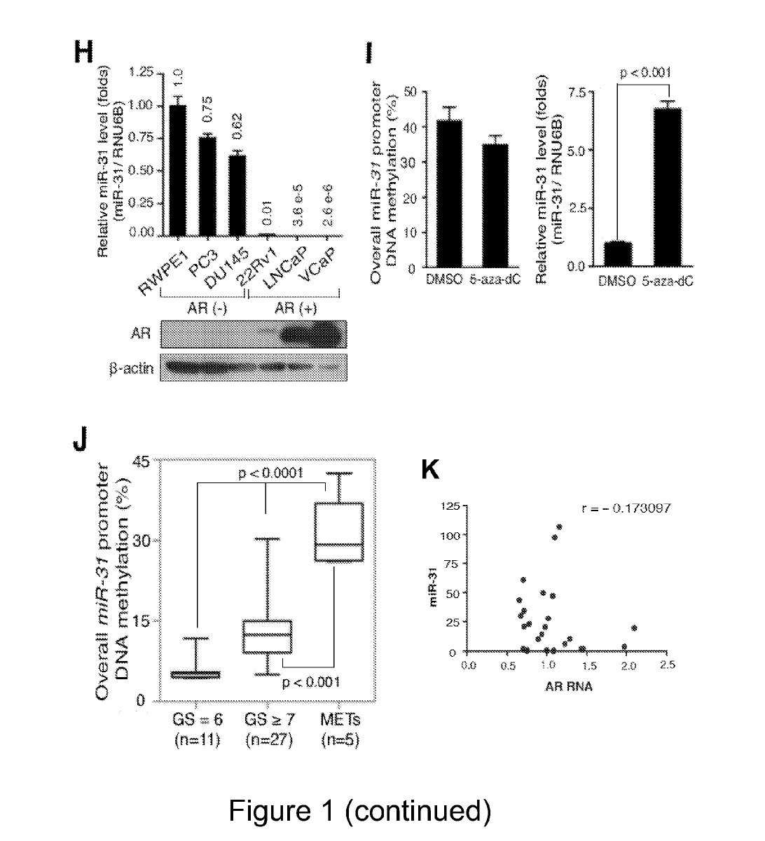

Here, the inventors interrogated 21 pairs of primary PCA and matched benign prostate tissue. 105 miRNAs were identified as significantly altered in PCA (FDR-adjusted p value <0.05, Table 1), including 25 miRNAs with at least 1.5-fold expression change (FIG. 1A; Table 2). The data showed upregulation of miR-182 and miR-375 and downregulation of miR-31, miR-145, miR-205, miR-221, and miR-222 in PCA.

The inventors then verified miR-31 expression in 14 of the 21 matched pairs, and 93% (13/14) showed decreased miR-31 expression in PCA with respect to matched benign prostate tissue (FIG. 1B). MiR-31 is located in the intronic region of its host gene MIR31HG (RefSeq NR_{—}027054). The overall expression of miR-31 and MIR31HG in a cohort of 40 primary PCA specimens was significantly lower as compared to 15 benign prostate tissues (p-value <0.0001, FIG. 1C). Taken together, our data demonstrated the downregulation of miR-31 in primary PCA.

### Example 2

### PCA-Specific Downregulation of miR-31 is Mediated by Promoter Hypermethylation

To delineate the mechanism behind the downregulation of miR-31 in PCA, the inventors first examined whether genomic (i.e., somatic) loss was responsible. By examining somatic copy number alterations across a variety of tumor types the inventors found that PCA did not have any deletion peaks at the MIR31HG locus (FIG. 1D). The genomic area spanning the MIR31HG locus and adjacent genes was deleted in only a small fraction (2-4%) of individuals with localized PCA. Altogether, the low rate of somatic copy number losses shows that genomic loss does not account for the high frequency of miR-31 downregulation in PCA.

Next, the inventors examined if epigenetic alterations account for the down-regulation of miR-31 expression. To determine, the inventors evaluated DNA methylation of the promoter region of MIR31HG/miR-31 on 12 matched samples by a direct quantitative DNA methylation assay (MassARRAY EpiTyping), with four pairs of primers (FIG. 1F; Table 3). The results revealed that the miR-31 promoter showed cancer-specific hypermethylation (p-value <0.001, FIG. 1E). PCA samples that displayed significantly higher levels of promoter methylation as compared to matched benign prostate tissues had lower miR-31 levels (ratio <1.0 in FIG. 1B). DNA methylation levels between PCA and benign prostate tissue were significantly different across the whole region (p-value <0.001) as well as in each of the four subdivided regions (p-values <0.006) (Table 4). Furthermore, three of individual CpG units showed cancer-specific DNA methylation changes (p-values <0.05). Taken together, DNA methylation levels at the miR-31 promoter were inversely correlated with miR-31 expression, revealing that promoter hypermethylation accounts for miR-31 downregulation in PCA.

The inventors then examined benign prostate and PCA cell lines for promoter hypermethylation and expression of miR-31. The immortalized human prostate epithelial cell line, RWPE1, and human PCA cell lines, PC3 and DU145, had high expression of miR-31 with little DNA methylation at the miR-31 promoter. In contrast, 22Rv1, LNCaP, LNCaP-abl, and VCaP cancer cells had low expression of miR-31 with concurrent high DNA methylation levels at the miR-31 promoter, consistent with what was observed in primary PCAs (FIG. 1G-H). Importantly, VCaP cells treated with the DNA methylation inhibitor 5-aza-2′-deoxycytidine (5-aza-dC) showed decreased DNA methylation levels at the miR-31 promoter and increased expression of miR-31 (FIG. 1I), supporting the role of promoter hypermethylation in downregulating miR-31 expression in PCA.

### Example 3

### MiR-31 Promoter Hypermethylation Correlates with Aggressiveness of PCA

The inventors then elucidated an association between miR-31 promoter methylation and PCA disease progression. PCA is graded using the Gleason score. A Gleason score ranges from 2-10 and higher scores (i.e. 7-10) are associated with a more aggressive clinical course. Thirty eight (38) primary PCA cases with Gleason scores ranging from 6 to 9 were examined along with 5 metastatic castration resistant PCA cases from patients who failed endocrine therapy and/or developed a predominantly androgen independent PCA associated with lack of AR expression and extensive neuroendocrine differentiation (Gleason scores are not assigned to metastatic PCAs). DNA methylation at the miR-31 promoter was positively correlated with PCA progression (Table 5). The overall DNA methylation at the miR-31 promoter showed significant differences among three groups: Gleason scores 6, ≧7, and metastatic cancer (FIG. 1J), and it was inversely correlated with miR-31 expression levels. Thus, this data demonstrated a close association between the extent of DNA methylation at the miR-31 promoter and the aggressiveness of PCA, and both promoter hypermethylation and downregulation of miR-31 serve as indicators for aggressive behaviors in PCA. This information will be useful to clinicians when evaluating patient prognosis and in directing therapeutic treatment.

### Example 4

### AR and H31 (27 Trimethylation Negatively Regulate miR-31 Expression

The Inventors then sought to identify other factors that could regulate miR-31 expression and found that AR expression levels were also inversely correlated with miR-31 expression levels in the prostate cell lines (FIG. 1H) and in primary PCA (r=−0.173097, p<0.42, FIG. 1K). AR-positive cells expressed much lower miR-31. VCaP cells with AR amplification and the highest AR expression showed the lowest expression level of miR-31. Activation of AR signaling with synthetic androgen (R1881), led to increasing expression of AR-targeting genes, NDRG1, PSA, and TMPRSS2, and downregulation of miR-31, while knocking down AR by siRNA interference reversed the repression on miR-31 (FIG. 2A). Additionally, PC3AR cells, which are PC3 cells engineered to express AR, and HEK293 cells transiently overexpressing AR also showed a decreased expression of miR-31 (FIG. 2B). Chromatin immunoprecipitation (ChIP) assays in LNCaP cells showed AR enrichment at the miR-31 promoter after androgen treatment, indicating a direct regulation of miR-31 expression by AR (FIG. 2C). To evaluate the binding of AR to the miR-31 promoter, luciferase assays were conducted by using the miR-31 promoter-driven luciferase reporter system. Expression of AR in HEK293 cells resulted in the inhibition of luciferase activity with constructs containing regions of the miR-31 promoter, showing that AR associates with the miR-31 promoter and inhibit its expression (FIG. 2D).

EZH2, is a methyltransferase involved in epigenetic silencing through H3K27 trimethylation (H3K27me3) that can negatively regulate the expression of miR-31. Inventors found that H3K27me3 was steadily enriched at the mIR-31 promoter and regions near miR-31 while EZH2 was recruited to these regions after androgen stimulation (FIG. 2C). Knocking down AR and EZH2 alone or simultaneously in LNCaP cells increased miR-31 expression, revealing that AR and EZH2 concurrently regulate the expression of miR-31 (FIG. 2E). Collectively, these data suggest that AR binding and repressive H3K27me3 coexist with promoter hypermethylation to downregulate miR-31 expression.

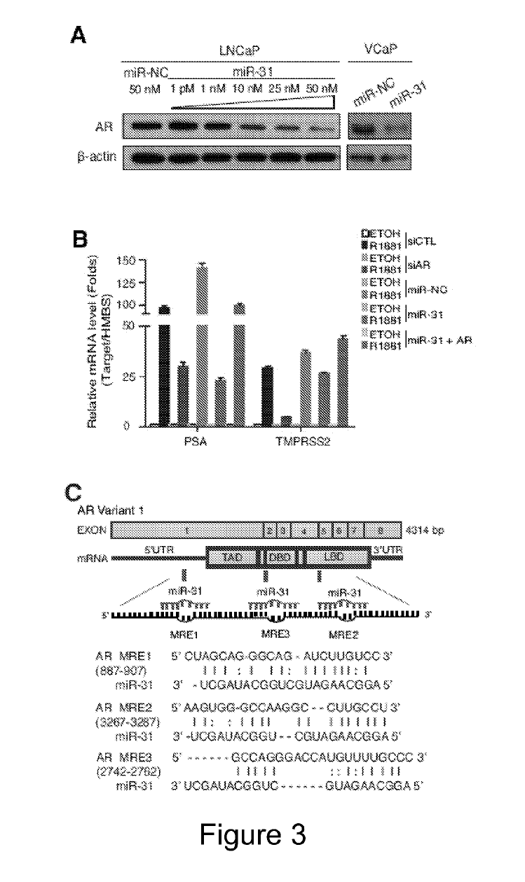

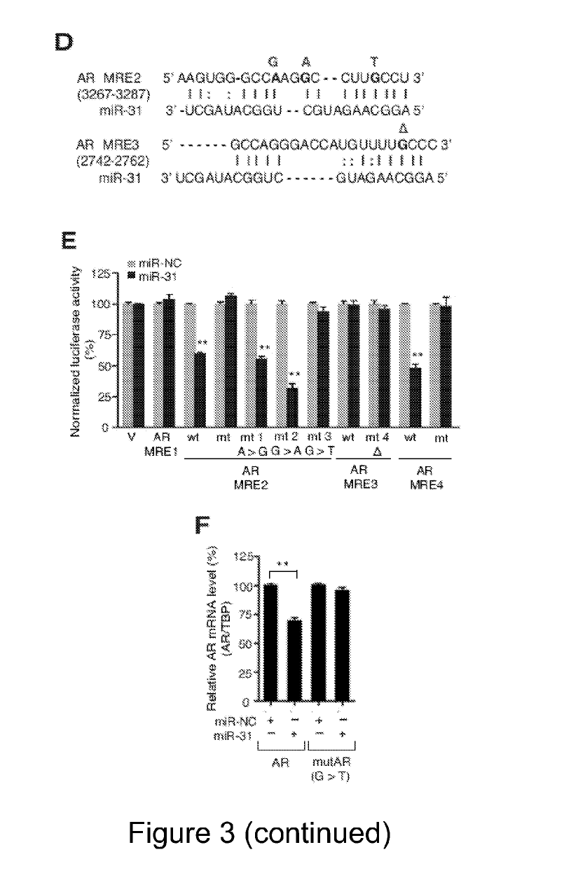

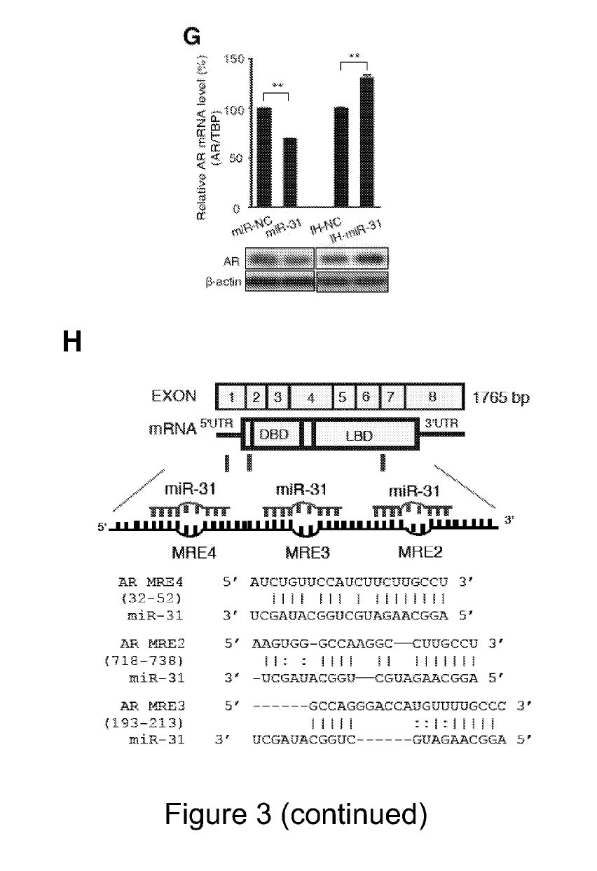

MiR-31 represses AR expression by targeting AR directly. LNCaP and VCaP cells transfected with increasing amounts of miR-31 showed decreased expression of AR at both the transcript and protein levels (FIG. 3A). qPCR assays also showed that miR-31 suppressed AR signaling, which was abrogated by overexpression of AR (FIG. 3B). While miRNA target-prediction algorithms provided by TargetScan, microRNA.org and PicTar did not list AR as a miR-31 target, we identified four putative miRNA recognition elements (MREs) of AR transcript variant 1 (RefSeq NM_{—}000044) and transcript variant 2 (RefSeq NM_{—}001011645) by RNA22 (23) (FIG. 3C and FIG. 3H). AR MRE1 and MRE4 were located at the 5′UTRs of AR variant 1 and 2, respectively. AR MRE2 and MRE3 were located at the coding sequence (CDS): MRE2 in the ligand-binding domain and MRE3 near the DNA binding domain, indicating that these sites may be important in regulating AR.

To determine whether reduced AR expression was directly mediated by miR-31, the inventors cloned the four predicted wild-type (WT) MREs as well as four mutations bearing MREs into a luciferase reporter system and performed co-transfection with either miR-31 or a negative control miR-NC in LNCaP cells (FIG. 3E) Inhibition of luciferase activity was shown with constructs containing MRE2 and MRE4 but not with constructs containing MRE1 or MRE3. Resistance to miR-31 repression was observed as a result of one of the three mutations at MRE2 (G>T), showing that this mutation leads to loss of AR regulation by miR-31. As MRE3 was not a bona fide target site for miR-31, the deletion at MRE3 had no effect on luciferase activity. Consistently, inhibition of AR expression by miR-31 occurred in 293HEK cells transfected with the construct containing the entire CDS of WT AR, but not the mutant construct (FIG. 3F). PC3AR cells, expressing the AR coding region and consequently MRE2, showed reduced AR expression upon overexpression of miR-31, while the miR-31 inhibitor increased AR expression (FIG. 3G). These results show that miR-31 can directly repress AR expression through the AR CDS.

### Example 5

### Genes Involved in Cell Cycle Regulation are Direct Targets of miR-31

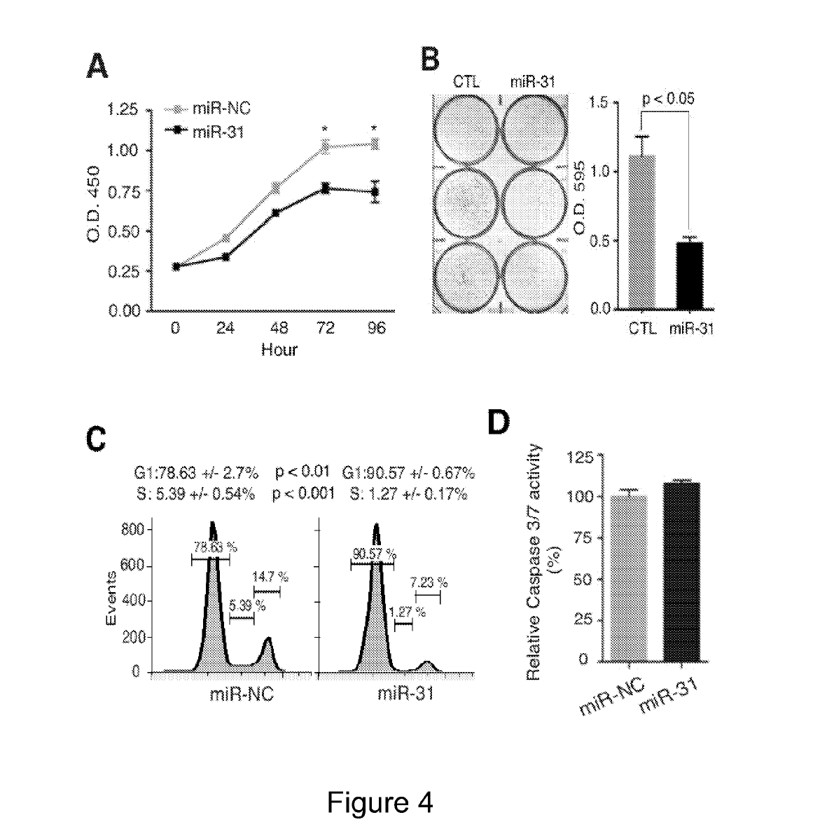

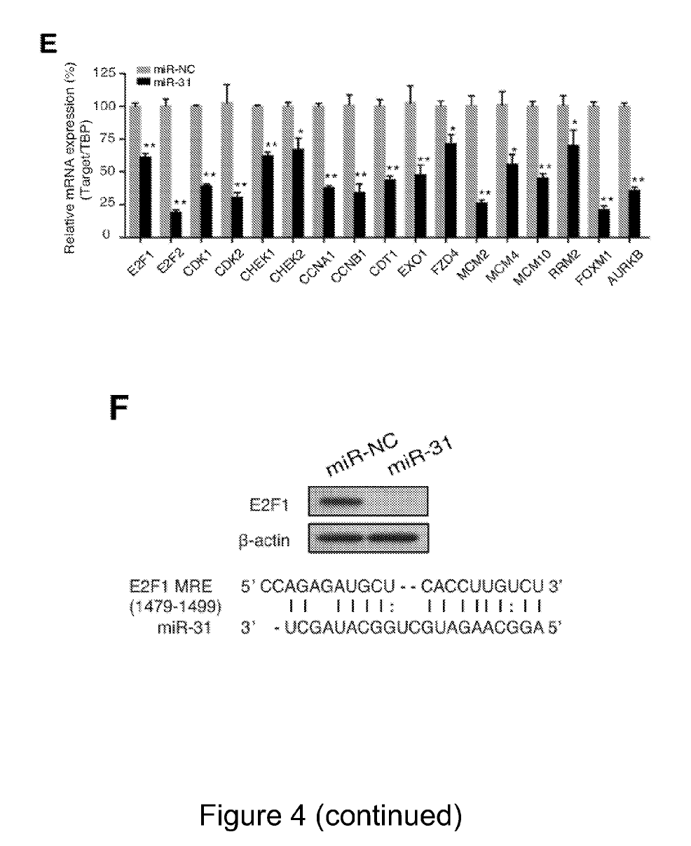

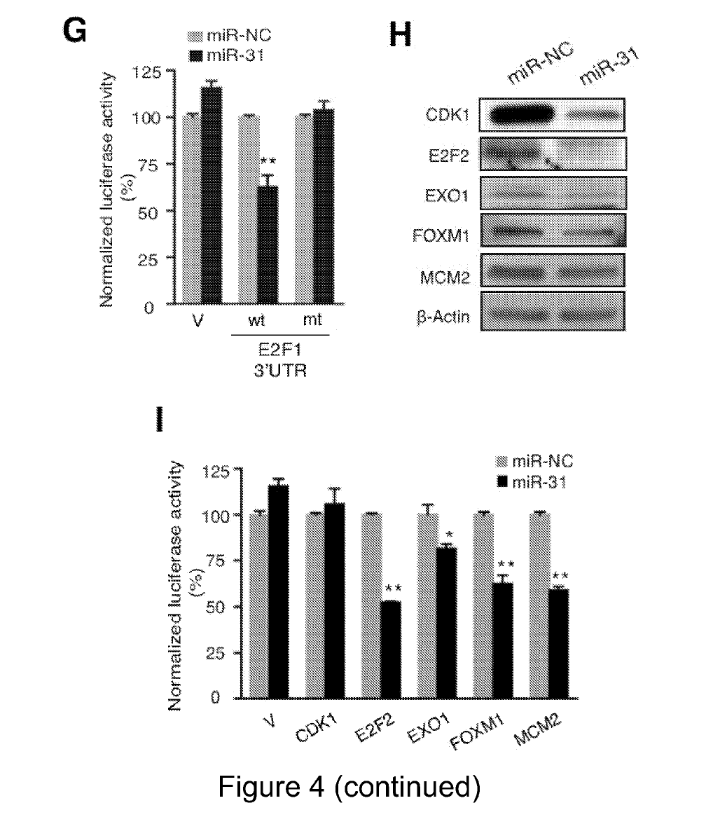

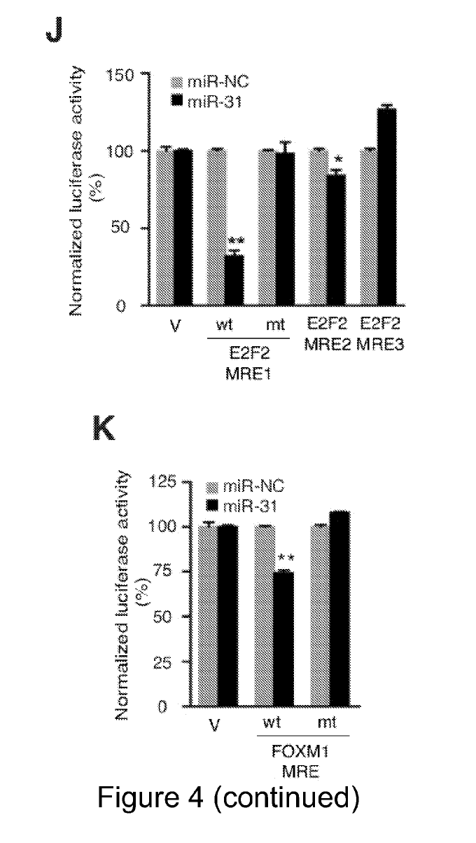

To gain insight into the cellular mechanism through which miR-31 exerts its effect, the inventors analyzed whole genome gene expression data from miR-31-overexpressing experiments in LNCaP cells. The top cellular processes that were enriched by gene ontology analysis (GO) included cell cycle, mitosis, DNA replication, microtubule-based process, and DNA repair (Table 6). Consistent with this analysis, overexpression of miR-31 inhibited cell proliferation and colony formation, and arrested cell cycle progression (FIG. 4A to 4C). The decreased cell proliferation was due to cell cycle arrest, since little apoptosis was observed as indicated by a minimal change in caspase-3/7 activity (FIG. 4D).

Expression levels of several genes involved in cell cycle regulation were decreased in the presence of miR-31 (FIG. 4E). Among them, transcription factor E2F1, which regulates AR expression, was decreased at both transcript and protein levels (FIG. 4F). One putative miR-31 MRE was identified at the 3′UTR of E2F1 Inhibition of luciferase activity was observed in cells expressing the WT construct, but not with the mutant (FIG. 4G), confirming that miR-31 targets E2F1 directly. These data suggested that miR-31 could regulate AR through direct repression of E2F1, in addition to directly targeting the AR mRNA.

The inventors also identified putative miR-31 MREs at 3′UTRs of CDK1, E2F2, EXO1, FOXM1, and MCM2, which are critical genes involved in cell cycle regulation (FIG. 4N). The transcript and protein levels of these genes were decreased in the presence of miR-31 (FIGS. 4E and 4H). Additionally, luciferase reporter assays were used to show that miR-31 directly represses the expression of E2F2, EXO1, FOXM1, and MCM2, but not CDK1 (FIG. 4I to 4M).

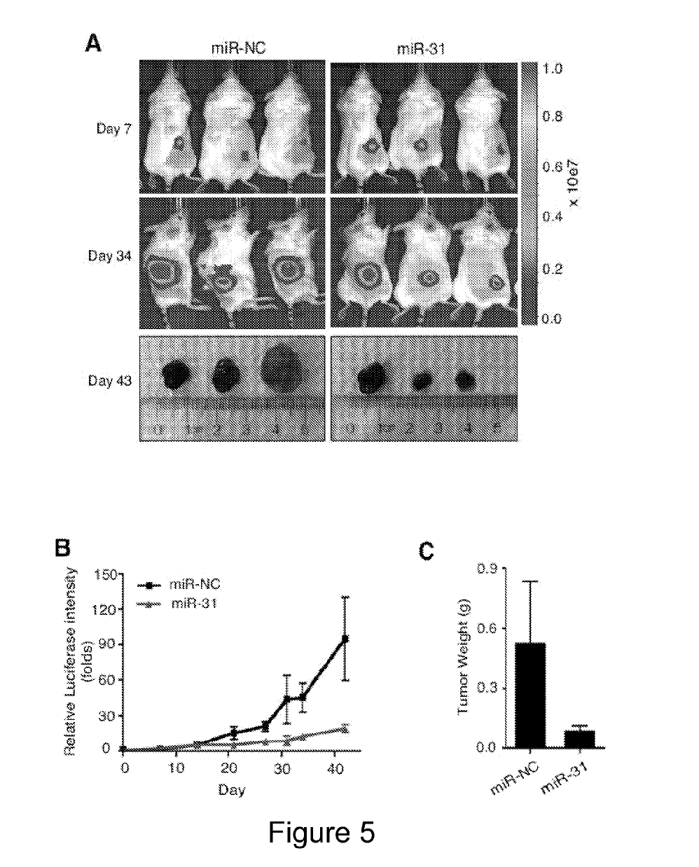

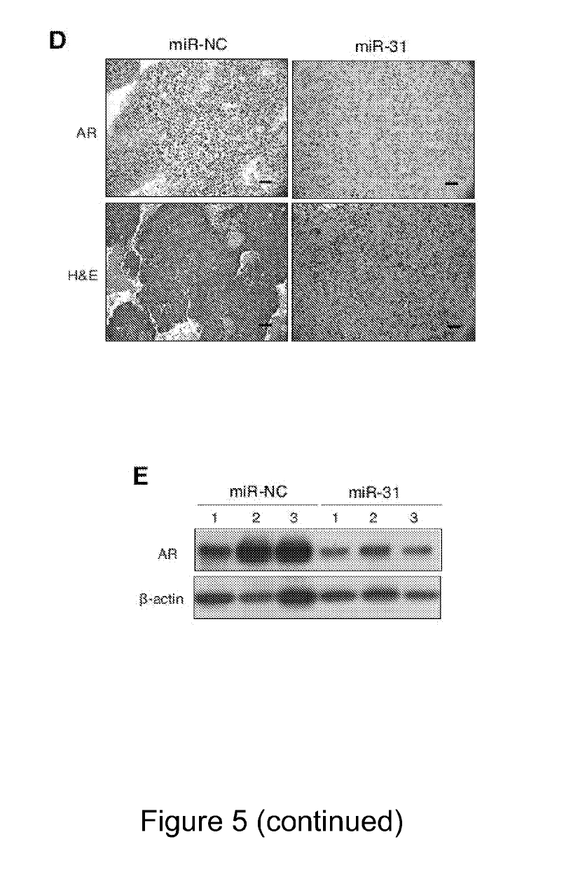

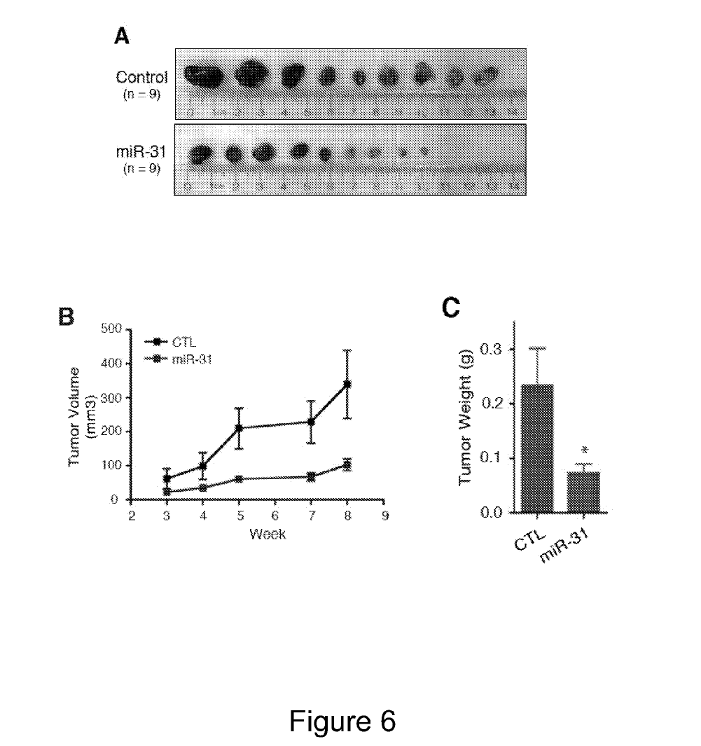

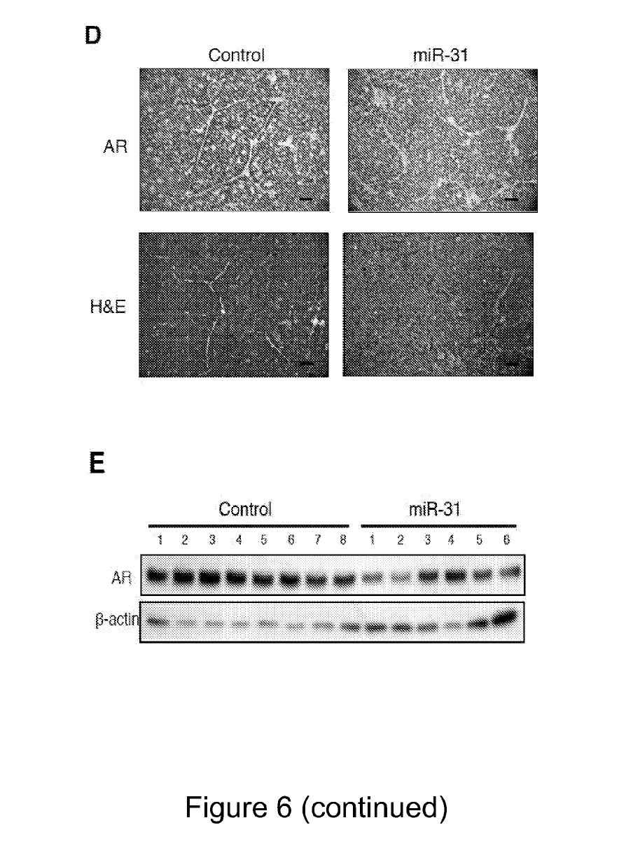

Example 6. MiR-31 represses PCA growth. To evaluate the anti-tumor effect of miR-31 in vivo, the inventors established murine xenograft experiments with LNCaP cells and treated tumors with miR-31 or control miR-NC mimics. Consistent with the in vitro data, miR-31 attenuated tumor growth over time (FIG. 5A to 5C). Additionally, tumors treated with miR-31 showed a marked reduction in AR expression (FIGS. 5D and 5E). Xenografts established with VCaP cells expressing miR-31 also showed smaller tumor sizes, decreased growth rates, and reduced AR levels. These data reveal that miR-31 represses PCA growth through the downregulation of AR (FIG. 6).

Example 7. Materials and Methods. Benign and PCA tissue selection. Hematoxylin and eosin (H&E) slides were prepared from frozen tissue blocks and evaluated for cancer extent and tumor grade by a pathologist and 1.5 mm biopsy cores of desired regions were taken from frozen tissue blocks for RNA/DNA extraction. These studies utilized tissues from clinically localized PCA patients who underwent radical prostatectomy as a primary therapy. The collection of samples from castration resistant metastatic PCA patients was carried according to previously known methods (see e.g. Beltran H, et al. Cancer Discov. (2011). 1:487-95). In brief, 1.5 mm biopsy cores were taken according to selection of high-density cancer foci (<10% stromal or other non-tumor tissue contamination) and benign regions, for RNA extraction by using TRIzol Reagent (Life technologies) or DNA extraction by using phenol-chloroform and purified by ethanol precipitation method as previously described (see Berger M F, et al. Nature. (2011). 470:214-20). RNA extracts were subjected to DNase treatment using a DNA-free™ Kit (Life technologies). The quality of RNA was assessed using the RNA 6000 Nano Kit on Bioanalyzer 2100 (Agilent). RNA with RIN (RNA integrity number)≧7 was used.

MiRNA Profiling.

Total RNA (100 ng) from each sample was run with GeneChip miRNA Array (Affymetrix). The two-sample Wilcoxon rank-sum test was applied to evaluate the difference between PCA and benign tissues. False discovery rate (FDR) control was used in multiple hypotheses testing to correct for multiple comparisons. miRNAs with significant changes were chosen based on adjusted p-value <0.05. To make the selection more stringent, fold change more than 1.5 and difference more than 100 were applied.

Quantitative DNA Methylation Analysis by MassARRAY EpiTyping.

Measurement of DNA methylation levels was performed by matrix-assisted laser desorption ionization/time-of-flight (MALDI-TOF) mass spectrometry using EpiTYPER assays by MassARRAY (Sequenom) on bisulfate-converted DNA according to the manufacturer's protocol. Bisulfite conversion was performed using EZ DNA methylation kit (Zymo Research). Additionally, varying reaction conditions and techniques using bisulfate are incorporated within the scope of this disclosure, including 3.1 M NaHSO3, 0.5 mM hydroquinone, pH 5.0, 50° C., 16 h or 40 h (see Frommer, M., et al. Proc. Natl. Acad. Sci. U.S.A., 80 (1992), 1579-1583); 3 M NaHSO3, pH 5, 50° C., 4 h (see Raizis, A. M., et al. Anal. Biochem., 226 (1995) 161-166); 5.36 M NaHSO3, 3.44 M urea, 0.5 mM hydroquinone, pH 5 (see Paulin, R., et al. Nucleic Acids Res., 26 (1998) 5009-5010); 4.75 M NaHSO3, pH 5.0, 50° C., 12-16 h (see Eads, C. A., et al. Methods Mol. Biol., 200 (2002) 71-85); 3.4 M NaHSO3, 1 mM hydroquinone, pH 5, 55° C., 6 h (see Laird, C. D. et al., Proc. Natl. Acad. Sci. U.S.A., 101 (2004) 204-209); and 9 M Bisufitea, pH 5.4, 70°, 40 min (see Shiraishi, M., et al. DNA Res., 11 (2004) 409-415). The spectra's methylation ratios were then calculated using EpiTYPER software v1.0 (Sequenom). EpiTYPER primers were designed through Sequenom EpiDesigner website http://www.epidesigner.com/start3.html.

| Name | Forward primer | Reverse primer |
|---|---|---|
| miR-31 CpG | aggaagagagTTTT | CAGTAATACGACTC |
| region 1 | TTTTAAGAAGGGAA | ACTATAGGGAGAAG |
| | AGTTTAG [SEQ | GCTCAAATAAACTA |
| | ID NO: 106]. | AAAAAACCTTAATC |
| | | CC [SEQ ID NO: |
| | | 107]. |
| miR-31 CpG | aggaagagagTTTT | CAGTAATACGACTC |
| region 2 | TAGGAGGAGTTTGG | ACTATAGGGAGAAG |
| | TGAGTAG [SEQ | GCTAAACCTCCTCA |
| | ID NO: 108]. | ACTCCTTAAAA |
| | | [SEQ ID NO: |
| | | 109]. |
| miR-31 CpG | aggaagagagGTTG | CAGTAATACGACTC |
| region 3 | TAGTGGAGAAATTT | ACTATAGGGAGAAG |
| | GGGTTT [SEQ ID | GCTCTCACCAAACT |
| | NO: 18]. | CCTCCTAAAAA |
| | | [SEQ ID NO: |
| | | 19]. |
| miR-31 CpG | aggaagagagTGGT | CAGTAATACGACTC |
| region 4 | TTTTGTAGGTGGAT | ACTATAGGGAGAAG |
| | TTTTTT [SEQ ID | GCTTACTAAACCTC |
| | NO: 20]. | CTCCCTTAAACCC |
| | | [SEQ ID NO: |
| | | 21]. |

For the association between Gleason score and DNA methylation, the average of grade % (outcome variable) was calculated for each primer; standard descriptive summary was then performed for this grade % average stratified by three categories (Gleason score=6, ≧7, and metastatic PCA). Kruskal-Wallis nonparametric test or Wilcoxon rank-sum test was used to evaluate the difference among three or between two of the three categories, respectively. In addition, Wilcoxon Sign Rank test was used to evaluate the difference for the paired samples. Adjusted p-value was calculated by Bonferoni Stepdown correction.

Methylated DNA Immunoprecipitation (MeDIP).

Isolated methylated DNA fragments are identified and procured by immunoprecipitating with 5′-methylcytosine-specific antibodies. The enriched methylated DNA is then analyzed in a locus-specific manner using PCR assay or in a genome-wide fashion by comparative genomic hybridization against a sample without MeDIP enrichment. (see e.g. Vuviv, E. A., et al. Microarray Analysis of the Physical Genome Methods in Molecular Biology, (2009) 556: 141-153)

Quantitative Real-Time PCR (qPCR).

cDNA synthesis was carried out using the M-MuLV Reverse Transcriptase (Emzymatics) according to the manufacturer's protocol. Quantitative real-time PCR was performed with the Roche LightCycler480 with SYBR Green I Master Mix or Probe Master Mix for Taqman Assay (Roche). Each sample was run in triplicate for every experiment. Taqman MicroRNA Assays (Life technologies) were used to quantify mature miRNA expression, carried out with Taqman MicroRNA Reverse Transcription Kit, hsa-miR-31 (AB Assay ID: 002279), and RNU6B (AB Assay ID: 001093) according to the manufacturer's protocol. Taqman gene expression assays (Life technologies) were carried out for E2F1, E2F2, CDK1, CDK2, CHEK1, CHEK2, CCNA1, CCNB1, CDT1, EXO1, FZD4, MCM2, MCM4, MCM10, RRM2, FOXM1, AURKB and TBP.

qPCR mRNA primers

| Name | Forward primer | Reverse primer |
|---|---|---|
| MIR31HG | CGCTTCTGTCCTCC | ACAAGCAGACCCTT |
| | TACTCG [SEQ ID | GGAATG [SEQ ID |
| | NO: 22]. | NO: 23]. |
| PSA | TGTGTGCTGGACGC | CACTGCCCCATGAC |
| | TGGA [SEQ ID | GTGAT [SEQ ID |
| | NO: 24]. | NO: 25]. |
| TMPRSS2 | GGACAGTGTGCACC | TCCCACGAGGAAGG |
| | TCAAAGAC [SEQ | TCCC [SEQ ID |
| | ID NO: 26]. | NO: 27]. |
| NDRG1 | GTGGAGAAAGGGGA | ACAGCGTGACGTGA |
| | GACCAT [SEQ ID | ACAGAG [SEQ ID |
| | NO: 28]. | NO: 29]. |
| HMBS | CCATCATCCTGGCA | GCATTCCTCAGGGT |
| | ACAGCT [SEQ ID | GCAGG [SEQ ID |
| | NO: 30]. | NO: 31]. |

To evaluate miR-31 expression levels under AR siRNA and R1881 stimulation, cells were transfected with 50 nM siRNA of AR or scrambled control and incubated in phenol-red free RPMI1640 media with 5% charcoal-stripped serum for 48 hours, and then stimulated with 1 nM R1881 for 24 hours.

Cell Line Development.

The benign prostate epithelial cell line, RWPE-1, and PCA cell lines, VCaP, LNCaP, 22Rv1, PC3, DU145, and HEK293 cells were purchased from American Type Culture Collection (ATCC) and used within 6 months after receipt; authentication of cell lines was performed by ATCC. PC3-neo and PC3-AR cell lines were characterized by short-tandem repeat profiling by Genetica DNA Laboratories Inc. and authenticated. Cells were maintained according to manufacturer and providers' protocols.

Small RNA Interference and miRNA Transfection.

Cells were treated with DharmaFECT2 transfection reagent (Dharmacon) for RNA interference and microRNA transfection, according to the manufacturer's protocol: non-targeting siRNA (D-001810-01), siRNA specific to EZH2 (11), AR (L-003400), miR-31 (C-300507-05), miR-31 inhibitor (IH-300507-06), miR mimic Negative Control/NC (CN-001000-01), and miR inhibitor NC (IN-001005-01).

Chromatin Immunoprecipitation (ChIP).

LNCaP cells were grown in phenol red-free RPMI 1640 media supplemented with 5% charcoal-stripped serum for 3 days, then treated with ethanol or 1 nM R1881 for 16-24 hours. Briefly, LNCaP cells were fixed using 1% formaldehyde for 10 minutes and quenched using 125 mM glycine for 5 minutes at room temperature, and then washed in ice-cold PBS twice. The cells were scrapped and centrifuged and the cell pellet was re-suspended in the dilution buffer (165 mM NaCl, 0.01% SDS. 1.1% Triton X-100, 1.2 mM EDTA pH 8.0, 16.7 mM Tris HCl pH8.0, 1 mM PMSF). Protein-bound chromatin was fragmented by sonication. Equal volumes of chromatin were immunoprecipitated with anti-AR, anti-EZH2, anti-trimethyl-Histone H3 Lys27 or normal IgG as a negative control (Millipore). Following extensive washing the DNA was eluted using 100 mM NaHCO3 and 1% SDS and the crosslinks were reversed using 300 mM NaCl at 65° C. for 16 hours. Immunoprecipitated DNA and whole cell extract DNA were purified by Qiaquick PCR purification kit (Qiagen). The purified DNA was amplified by real-time quantitative PCR with Roche SYBR Green PCR master mix and analyzed for enrichment. Real-time qPCR amplification was performed with Roche LightCycle480. (see Boyd K E, et al. Proc Natl Acad Sci USA. (1998). 95:13887-92). Sequences of ChIP primers are provided below:

| Name | Forward primer | Reverse primer |
|---|---|---|
| −1,000 | CCGATGACCTAGCC | CCCCACCCTTCAAC |
| bp | AGAAGT [SEQ ID | TCGTAG [SEQ ID |
| | NO: 32]. | NO: 33]. |
| −500 | TATCCTCAACCCTC | CATACACCTGAAGG |
| bp | CGTGTC [SEQ ID | GGCAGT [SEQ ID |
| | NO: 34]. | NO: 35]. |
| +500 | CAATTTTGGCCCAG | TTTCCGGGGACCTC |
| bp | GAGATA [SEQ ID | TAGTTT [SEQ ID |
| | NO: 36]. | NO: 37]. |
| +42,500 | TGGCCTATTTGCTG | GCAAGCCAACCCCA |
| bp | TTCTAATGAC | ACA [SEQ ID |
| | [SEQ ID NO: | NO: 39]. |
| | 38]. | |
| +45,000 | AATGGGCCCTGCAT | AAAACCCACACCCT |
| bp | TCTCT [SEQ ID | CACCAC [SEQ ID |
| | NO: 40]. | NO: 41]. |
| +47,500 | CATCTTCAAAAGCG | ACAATACATAGCAG |
| bp | GACACTCT [SEQ | GACAGGAAG [SEQ |
| | ID NO: 42]. | ID NO: 43]. |
| PSA | CCTAGATGAAGTCT | GGGAGGGAGAGCTA |
| | CCATGAGCTACA | GCACTTG [SEQ |
| | [SEQ ID NO: | ID NO: 45]. |
| | 44]. | |
| MYT1 | AGGCACCTTCTGTT | AGGCAGCTGCCTCC |
| | GGCCGA [SEQ ID | CGTACA [SEQ ID |
| | NO: 46]. | NO: 47]. |
| GAPDH | CGGCTACTAGCGGT | AAGAAGATGCGGCT |
| | TTTACG [SEQ ID | GACTGT [SEQ ID |
| | NO: 48]. | NO: 49]. |

MiRNA Reporter Luciferase Assays.

LNCaP cells were transfected in triplicate with 30 nM miR-31 or control miRNA-NC mimic together with psiCHECK2 vector (Promega; 0.4 μg/well, 24-well plate) containing 21-bp MiRNA Recognition Elements (MREs) or the 3′UTR region containing the MREs of indicated genes by DharmaFECT Duo transfection reagent, according to the manufacturer's protocol (Dharmacon). After 48 hours, cells were lysed and luciferase activity was measured using the Dual Luciferase Assay System (Promega) and GloMax®-Multi Detection System (Promega). Data were normalized to Firefly luciferase. Individual wild type and mutant MREs were cloned into psiCHECK2 vector (see e.g. Lal A, et al. (Mol Cell. 2009). 35:610-25). psiCHECK2-E2F1 3′UTR (Addgene plasmid 29468). Site-directed mutagenesis was carried out by the QuikChange Site-Directed Mutagenesis Kit (Agilent). Sequences of primers used in the preparation of constructs.

| Name | Forward | Reverse |
|---|---|---|
| AR MRE1 | TCGAGCTAGCA | GGCCGCGGACA |
| | GGGCAGATCTT | AGATCTGCCCT |
| | GTCCGC [SEQ | GCTAGC [SEQ |
| | ID NO: 50]. | ID NO: 51]. |
| AR MRE2 | TCGAGAAGTGG | GGCCGCAGGCA |
| | GCCAAGGCCTT | AGGCCTTGGCC |
| | GCCTGC [SEQ | CACTTC [SEQ |
| | ID NO: 52]. | ID NO: 53]. |
| AR MRE3 | TCGAGGCCAGG | GGCCGCGGGCA |
| | GACCATGTTTT | AAACATGGTCC |
| | GCCCGC [SEQ | CTGGCC [SEQ |
| | ID NO: 54]. | ID NO: 55]. |
| AR MRE4 | TCGAGATCTGT | GGCCGCAGGCA |
| | TCCATCTTCTT | AGAAGATGGAA |
| | GCCTGC [SEQ | CAGATC [SEQ |
| | ID NO: 56]. | ID NO: 57]. |
| AR MRE2 | TCGAGAAGTGG | GGCCGCACCCT |
| mt | GCCAAGGCCAA | TGGCCTTGGCC |
| | GGGTGC [SEQ | CACTTC [SEQ |
| | ID NO: 58]. | ID NO: 59]. |
| AR MRE2 | TCGAGAAGTGG | GGCCGCAGGCA |
| mt1 | GCCGAGGCCTT | AGGCCTCGGCC |
| | GCCTGC [SEQ | CACTTC [SEQ |
| | ID NO: 60]. | ID NO: 61]. |
| AR MRE2 | TCGAGAAGTGG | GGCCGCAGGCA |
| mt2 | GCCAAGACCTT | AGGTCTTGGCC |
| | GCCTGC [SEQ | CACTTC [SEQ |
| | ID NO: 62]. | ID NO: 63]. |
| AR MRE2 | TCGAGAAGTGG | GGCCGCAGGAA |
| mt3 | GCCAAGGCCTT | AGGCCTTGGCC |
| | TCCTGC [SEQ | CACTTC [SEQ |
| | ID NO: 64]. | ID NO: 65]. |
| AR MRE3 | TCGAGGCCAGG | GGCCGCGGGAA |
| mt4 | GACCATGTTTT | AACATGGTCCC |
| | CCCGC [SEQ | TGGCC [SEQ |
| | ID NO: 66]. | ID NO: 67]. |
| AR MRE4 | TCGAGATCTGT | GGCCGCACCCA |
| mt | TCCATCTTGAA | ACAAGATGGAA |
| | GGGTGC [SEQ | CAGATC [SEQ |
| | ID NO: 68]. | ID NO: 69]. |
| CDK1 | ATGACTCGAGA | ATGAGCGGCCG |
| 3′UTR | CGAATTTCTGG | CGAGCCTTTTT |
| | CAAAATGG | AGATGGCTGCT |
| | [SEQ ID NO: | [SEQ ID NO: |
| | 70]. | 71]. |
| E2F2 | ATGACTCGAGC | ATGAGCGGCCG |
| 3′UTR | CCCCTCTACTG | CGGACCATGTT |
| | TCCCTTTC | CTCTCGGTTC |
| | [SEQ ID NO: | [SEQ ID NO: |
| | 72]. | 73]. |
| EXO1 | ATGACTCGAGT | ATGAGCGGCCG |
| 3′UTR | CCAGAAGCGGA | CCCTCCAAAAA |
| | AGAGGATA | CCTGTCAGAGA |
| | [SEQ ID NO: | [SEQ ID NO: |
| | 74]. | 75]. |
| FOXM1 | ATGACTCGAGC | ATGAGCGGCCG |
| 3′UTR | CCTGACAACAT | CTTTTTGTCCA |
| | CAACTGGTC | CCTTCGCTTT |
| | [SEQ ID NO: | [SEQ ID NO: |
| | 76]. | 77]. |
| MCM2 | ATGACTCGAGT | ATGAGCGGCCG |
| 3′UTR | TCCTTGGGATT | CCAGCCTGAAT |
| | CTGGTTTG | ACGCAACTCA |
| | [SEQ ID NO: | [SEQ ID NO: |
| | 78]. | 79]. |
| AR long | ATGACTCGAGT | ATGAGCGGCCG |
| 3′UTR | GGAGCCAGAGG | CGAGTTCATGG |
| | AGAAGAAA | GTGGCAAAGT |
| | [SEQ ID NO: | [SEQ ID NO: |
| | 80]. | 81]. |
| CDK1 MRE | TCGAGAGAGCA | GGCCGCTTAGC |
| | TGCCAAAATTT | AAATTTTGGCA |
| | GCTAAGC | TGCTCTC |
| | [SEQ ID NO: | [SEQ ID NO: |
| | 82]. | 83]. |
| E2F2 MRE1 | TCGAGAGCTCA | GGCCGCTGGCA |
| | TGCCCCCGTCT | AGACGGGGGCA |
| | TGCCAGC | TGAGCTC |
| | [SEQ ID NO: | [SEQ ID NO: |
| | 84]. | 85]. |
| E2F2 MRE1 | TCGAGACGACT | GGCCGCTCCGT |
| mt | ACGGTCGTAGA | TCTACGACCGT |
| | ACGGAGC | AGTCGTC |
| | [SEQ ID NO: | [SEQ ID NO: |
| | 86]. | 87]. |
| E2F2 MRE2 | TCGAGGTGAGC | GGCCGCAGGCA |
| | TGAAGAACCTT | AGGTTCTTCAG |
| | GCCTGC | CTCACC |
| | [SEQ ID NO: | [SEQ ID NO: |
| | 88]. | 89]. |
| E2F2 MRE3 | TCGAGGCAGCT | GGCCGCGGCAA |
| | GTGGGCCCTTT | AAGGGCCCACA |
| | TGCCGC [SEQ | GCTGCC [SEQ |
| | ID NO: 90]. | ID NO: 91]. |
| FOXM1 MRE | TCGAGGACAAG | GGCCGCCTGGC |
| | TGGATCTGCTT | AAGCAGATCCA |
| | GCCAGGC | CTTGTCC |
| | [SEQ ID NO: | [SEQ ID NO: |
| | 92]. | 93]. |
| FOXM1 | TCGAGGAGATG | GGCCGCCTCCG |
| MREmt | TGGTTGTGGAA | TTCCACAACCA |
| | CGGAGGC | CATCTCC |
| | [SEQ ID NO: | [SEQ ID NO: |
| | 94]. | 95]. |
| MCM2 MRE | TCGAGTGGTTG | GGCCGCTGGCA |
| | CTGAACATCTT | AGATGTTCAGC |
| | GCCAGC [SEQ | AACCAC [SEQ |
| | ID NO: 96]. | ID NO: 97]. |
| MCM2 MRE | TCGAGTGGTTG | GGCCGCTCCCT |
| mt | CTGAACATCAA | TGATGTTCAGC |
| | GGGAGC [SEQ | AACCAC [SEQ |
| | ID NO: 98]. | ID NO: 99]. |

Primers for Site-Directed Mutagenesis

| Name | Forward | Reverse |
|---|---|---|
| AR long | GGCAAGCTGGGCGTCT | CATCTCTGGGGGACAAGT |
| 3′UTR mut | TAAACTTGTCCCCCAG | TTAAGACGCCCAGCTTGC |
| | AGATG [SEQ ID | C [SEQ ID NO: 101] |
| | NO: 100] | |
| AR CDS | CAAGTGGGCCAAGGCC | GTAAGTTGCGGAAGCCAG |
| mut | TTTCCTGGCTTCCGCA | GAAAGGCCTTGGCCCACT |
| | ACTTAC [SEQ ID | TG [SEQ ID |
| | NO: 102] | NO: 103] |
| E2F1 | GTTTCCAGAGATGCTC | GCTCCAGGGCTGCAGAGA |
| 3′UTR mut | ACCAACTCTCTGCAGC | GTTGGTGAGCATCTCTGG |
| | CCTGGAGC [SEQ ID | AAAC [SEQ ID |
| | NO: 104] | NO: 105] |

For promoter binding, HEK293 cells were transfected in triplicate using Lipofectamine 2000 (Life technologies) with pCMV vector or pCMV-HA-AR expression vector together with the pGL3-IRES-promoter reporter constructs containing the MIR31HG promoter regions as indicated. pRL-TK vector for Renilla luciferase activity was used as internal control. The promoter regions were cloned in the NheI site by using In Fusion PCR cloning system (Clontech), according to manufacturer's instructions.

Primers for the Promoter Regions

| Name | Forward | Reverse |
|---|---|---|
| Promoter | TCTTACGCGTGCTAG | TCGAGCCCGGGCTAG |
| −1000 bp | CCCCCAAGTTATGCA | CCCCTTCAAATCCAG |
| | CAGGTC | GTGAAA |
| | [SEQ ID NO: 10] | [SEQ ID NO: 11] |
| Promoter | TCTTACGCGTGCTAG | TCGAGCCCGGGCTAG |
| +500 by | CTAAAGCAGCTGCCC | CCGAAGTCACAGGTT |
| | AATTTT | CGCTCT |
| | [SEQ ID NO: 12] | [SEQ ID NO: 13] |

Prostate Tumor Xenograft Model. Six to eight-week-old male CB17 SCIDs (defined flora, from Taconic) were used. For LNCaP xenografts, 1×106 viable single cells were resuspended in a 1:1 mixture of PBS and growth-factor-reduced Matrigel (BD Biosciences) and implanted subcutaneously, and after 4 weeks animals were randomly assigned to miR-31 and NC groups. LNCaP tumors were repeatedly injected intratumorally (see e.g. Wiggins J F, et al. Cancer Res. (2010). 70:5923-30) with miR-NC or miR-31 oligos formulated with MaxSuppressor In Vivo RNA-LANCEr II (BIOO Scientific) following the manufacturer's protocol, every 5 days for 6 times. The experiment was ended to 43 days after initiation of injections. Primary tumor growth rates were analyzed by measuring luciferase intensity according to the manufacturer's protocol (Caliper). On the day of imaging, D-Luciferin was intraperitoneally injected (150 mg/kg) into anesthetized mice. Bioluminescence images were acquired with the IVIS Imaging System (Caliper) 5-10 minutes post injection Analysis was performed using LivingImage software (Caliper). All tumors were sectioned for histological analysis. For VCaP xenografts, VCaP cells were transduced with lentivirus expressing miR-31 or empty vector and 0.2×106 cells were resuspended in a 1:1 mixture of PBS and growth-factor-reduced Matrigel (BD Biosciences) and subcutaneously implanted. The experiment was terminated 60 days after implantation. Tumor size was measured weekly, and tumor volumes were estimated using the formula (π⅙) (L×W2)(mm³), where L=length of tumor and W=width. All tumors were sectioned for histological analysis.

Data Analysis and Statistical Methods.

Statistical analysis of expression data was performed with GraphPad Prism 4.0 (Graph Pad software). Two-sided and p values <0.05 were considered statistically significant.

Immunoblot Analysis.

Cell pellets were flash frozen, briefly sonicated and homogenized in RIPA lysis buffer (Thermo Scientific) containing the halt protease inhibitor cocktail (Thermo Scientific). Lysates were briefly sonicated and cleared by centrifugation. Each protein extract was boiled in sampling buffer, size fractionated by SDS-PAGE, and transferred onto Polyvinylidene Difluoride membrane (Immobilon-P, Millipore). The membrane was then incubated overnight at 4° C. in blocking buffer [Tris-buffered saline, 0.1% Tween (TBS-T), 1% nonfat dry milk or 3% BSA] with the following antibodies: anti-AR (Millipore, 06-680), anti-EZH2 (BD, 612667), anti-E2F1 (Cell Signaling, 3742), anti-CDK1 (Cell Signaling, 9112), anti-MCM2 (Cell Signaling, 4007), anti-FOXM1 (Santa Cruz Biotechnology, sc-32855), anti-c-myc (Roche, 11667149001), anti-E2F2 (Sigma, E8776), EXO1 (Abcam, ab82533), and anti-β-Actin (Santa Cruz Biotechnology, sc-47778) antibodies Following extensive wash with TBS-T, the blot was incubated with horseradish peroxidase-conjugated secondary antibody and the signals detected by Luminata Western HRP substrates according to the manufacturer's protocol (Millipore).

Global Gene Expression Analysis.

Gene expression profiles were performed with the Illumina HumanHT12-v4 Expression BeadChip according to the manufacturer's protocol. LNCaP cells were transfected with 50 nM miR-31 or miR-NC mimics for 48 hours. Data reprocessing: the background subtracted and normalized (by Illumina Beadstudio) data was used for the analysis. All expression values with detection signals<0.95 were called NAs. Probes for which more than 50% of the samples had NAs were removed. The next steps applied for all pair-wise differential expression analysis. Fold change was calculated between the two groups. The raw expression values were averaged across the two replicates and log 2 of the fold change was calculated. The empirical distribution of the log 2FC was plotted and the 2.5% and 97.5% percentiles were used for cutoffs.

Proliferation Assay.

Two methods were applied to measure cell proliferation: Roche WST-1 and CellTiter-Glo™ Luminescent Cell Viability Assay (Promega), according to manufacturers' protocols. To measure the effects of miR-31 or miR-31 inhibitor on cell proliferation, LNCaP, PC3, RWPE1 cells were transfected with 50 nM miR-31 mimics, miR-31 inhibitor, miR-NC, inhibitor-NC using DharmaFECT2 (Dharmacon) following the manufacturer's protocol. 24 hours later cells were plated in replicates of 6 in 96-well plates and cell viability was measured as indicated.

Cell Cycle Analysis.

For cell cycle analysis, LNCaP or PC3 cells were transfected with miR-31, miR-31 inhibitor or miR-NC mimics as described above. After 72-hour transfection, cells were collected and stained with propidium iodide (Sigma) and analyzed by flow cytometry using the LSR-II Flow Cytometer system (BD) and FlowJo software following the manufacturers' protocols.

Apoptosis Analysis.

To measure the effects of miR-31 on apoptosis, LNCaP or PC3 cells were transfected with 50 nM miR-31 and miR-NC using DharmaFECT2 (Dharmacon) following the manufacturer's protocol. After 24-hour transfection, LNCaP cells were re-plated in replicates of 6 in 96-well plates and Caspase-3/7 activities were measured after another 48 hours using Caspase-Glo 3/7 assay (Promega). For Annexin V/PI assays, PC3 cells transfected with miR-31, miR-31 inhibitor, or miR-NC mimics were prepared by the Annexin V: FITC Apoptosis Detection Kit II (BD) and then monitored for apoptosis by flow cytometry in accordance with the manufacturer's protocols. Briefly, cells were washed twice with PBS and stained with 5 μl of Annexin V-FITC and 10 μl of PI (5 μg/ml) in 1× binding buffer (10 mM HEPES, pH 7.4, 140 mM NaOH, 2.5 mM CaCl2) for 15 min at room temperature in the dark. Apoptotic cells were quantified using a LSR-II Flow Cytometer system (BD). Both early apoptotic (Annexin V-positive, PI-negative) and late apoptotic (double positive of Annexin V and PI) cells were evaluated.

All microarray data are deposited in the GEO database under accession number GSE36803.

TABLE 1
List of 105 differentially expressed miRNAs

| | | |
|---|---|---|
| | | FDR-adjusted |
| No. | probe_ID | p-value |

Upregulation

| | | |
|---|---|---|
| 1 | hsa-miR-770-5p_st | 0.00005 |
| 2 | hsa-miR-93_st | 0.00008 |
| 3 | hsa-miR-936_st | 0.00008 |
| 4 | hsa-miR-153_st | 0.0001 |
| 5 | hsa-miR-25_st | 0.0001 |
| 6 | hsa-miR-1207-5p_st | 0.00018 |
| 7 | hsa-miR-182_st | 0.00022 |
| 8 | hsa-miR-141_st | 0.00022 |
| 9 | hsa-miR-665_st | 0.00022 |
| 10 | hsa-miR-425_st | 0.00056 |
| 11 | hsa-miR-768-5p_st | 0.00067 |
| 12 | hsa-miR-1291_st | 0.00085 |
| 13 | hsa-miR-1224-5p_st | 0.00127 |
| 14 | hsa-miR-375_st | 0.00157 |
| 15 | hsa-miR-130b_st | 0.00187 |
| 16 | hsa-miR-200c_st | 0.00187 |
| 17 | hsa-miR-551b-star | 0.00218 |
| 18 | hsa-miR-768-3p_st | 0.00218 |
| 19 | hsa-miR-106b_st | 0.0026 |
| 20 | hsa-miR-17_st | 0.0026 |
| 21 | hsa-miR-96_st | 0.00387 |
| 22 | hsa-miR-148a-star | 0.00447 |
| 23 | hsa-miR-93-star_s | 0.00447 |
| 24 | hsa-miR-769-5p_st | 0.00592 |
| 25 | hsa-miR-17-star_s | 0.00592 |
| 26 | hsa-miR-20a_st | 0.00592 |
| 27 | hsa-miR-512-3p_st | 0.00592 |
| 28 | hsa-miR-921_st | 0.00592 |
| 29 | hsa-miR-7-1-star_st | 0.0078 |
| 30 | hsa-miR-148a_st | 0.0078 |
| 31 | hsa-miR-7-2-star_st | 0.0078 |
| 32 | hsa-miR-1273_st | 0.0078 |
| 33 | hsa-miR-625-star_st | 0.0078 |
| 34 | hsa-miR-106a_st | 0.0078 |
| 35 | hsa-miR-1179_st | 0.01061 |
| 36 | hsa-miR-7_st | 0.01193 |
| 37 | hsa-miR-191_st | 0.01494 |
| 38 | hsa-miR-200c-star_st | 0.01607 |
| 39 | hsa-miR-1201_st | 0.01728 |
| 40 | hsa-miR-558_st | 0.01856 |
| 41 | hsa-miR-542-3p_st | 0.02317 |
| 42 | hsa-miR-484_st | 0.02449 |
| 43 | hsa-miR-370_st | 0.02449 |
| 44 | hsa-miR-1280_st | 0.02607 |
| 45 | hsa-miR-107_st | 0.0264 |
| 46 | hsa-miR-1244_st | 0.02679 |
| 47 | hsa-miR-1285_st | 0.02679 |
| 48 | hsa-miR-720_st | 0.0377 |
| 49 | hsa-miR-18b_st | 0.03893 |
| 50 | hsa-miR-1254_st | 0.03893 |
| 51 | hsa-miR-449a_st | 0.04397 |
| 52 | hsa-miR-32_st | 0.04953 |

Downregulation

| | | |
|---|---|---|
| 53 | hsa-miR-139-5p_st | 0.00005 |
| 54 | hsa-miR-205_st | 0.00005 |
| 55 | hsa-miR-27b_st | 0.0001 |
| 56 | hsa-miR-221_st | 0.0001 |
| 57 | hsa-miR-222_st | 0.0001 |
| 58 | hsa-miR-376c_st | 0.00012 |
| 59 | hsa-miR-145-star_st | 0.00012 |
| 60 | hsa-miR-133a_st | 0.00012 |
| 61 | hsa-miR-143-star_st | 0.00016 |
| 62 | hsa-miR-455-3p_st | 0.00018 |
| 63 | hsa-miR-125a-5p_st | 0.00018 |
| 64 | hsa-miR-145_st | 0.00018 |
| 65 | hsa-miR-133b_st | 0.00022 |
| 66 | hsa-miR-152_st | 0.00067 |
| 67 | hsa-miR-31_st | 0.00067 |
| 68 | hsa-miR-181c_st | 0.00084 |
| 69 | hsa-miR-593_st | 0.00105 |
| 70 | hsa-miR-224_st | 0.00105 |
| 71 | hsa-miR-204_st | 0.00127 |
| 72 | hsa-miR-221-star_st | 0.00127 |
| 73 | hsa-miR-508-3p_st | 0.00157 |
| 74 | hsa-miR-181a_st | 0.00187 |
| 75 | hsa-miR-621_st | 0.00218 |
| 76 | hsa-miR-24_st | 0.00218 |
| 77 | hsa-miR-455-5p_st | 0.0026 |
| 78 | hsa-miR-320a_st | 0.00387 |
| 79 | hsa-miR-149_st | 0.00387 |
| 80 | hsa-miR-505_st | 0.00447 |
| 81 | hsa-miR-23b_st | 0.00447 |
| 82 | hsa-miR-1_st | 0.00506 |
| 83 | hsa-miR-23a_st | 0.00506 |
| 84 | hsa-miR-100_st | 0.00592 |
| 85 | hsa-miR-886-3p_st | 0.0067 |
| 86 | hsa-miR-34a-star_st | 0.00702 |
| 87 | hsa-miR-24-1-star_st | 0.00931 |
| 88 | hsa-miR-27a_st | 0.01029 |
| 89 | hsa-miR-30c_st | 0.01044 |
| 90 | hsa-miR-99b_st | 0.01061 |
| 91 | hsa-miR-378_st | 0.01105 |
| 92 | hsa-miR-502-5p_st | 0.01193 |
| 93 | hsa-miR-31-star_s | 0.01193 |
| 94 | hsa-miR-143_st | 0.01193 |
| 95 | hsa-miR-422a_st | 0.01404 |
| 96 | hsa-miR-222-star_st | 0.01607 |
| 97 | hsa-miR-379_st | 0.02449 |
| 98 | hsa-miR-28-3p_st | 0.02679 |
| 99 | hsa-miR-30e-star_st | 0.02679 |
| 100 | hsa-miR-1287_st | 0.03054 |
| 101 | hsa-miR-181d_st | 0.03054 |
| 102 | hsa-miR-320c_st | 0.03506 |
| 103 | hsa-miR-130a_st | 0.03893 |
| 104 | hsa-miR-891b_st | 0.04397 |
| 105 | hsa-miR-886-5p_st | 0.04953 |

TABLE 2
Table 2, List of 25 miRNAs ≧ 1.5 fold expression change

| | | FDR-adjusted | Fold |
|---|---|---|---|
| No. | probe_ID | p-value | changes |
| 1 | hsa-miR-182_st | 0.00022 | 3.164 |
| 2 | hsa-miR-375_st | 0.00157 | 2.769 |
| 3 | hsa-miR-25_st | 0.0001 | 2.480 |
| 4 | hsa-miR-93_st | 0.00008 | 2.192 |
| 5 | hsa-miR-141_st | 0.00022 | 2.041 |
| 6 | hsa-miR-148a_st | 0.0078 | 2.016 |
| 7 | hsa-miR-665_st | 0.00022 | 1.952 |
| 8 | hsa-miR-106b_st | 0.0026 | 1.820 |
| 9 | hsa-miR-425_st | 0.00056 | 1.807 |
| 10 | hsa-miR-1291_st | 0.00085 | 1.791 |
| 11 | hsa-miR-200c_st | 0.00187 | 1.784 |
| 12 | hsa-miR-20a_st | 0.00592 | 1.699 |
| 13 | hsa-miR-1224-5p_st | 0.00127 | 1.571 |
| 14 | hsa-miR-1207-5p_st | 0.00018 | 1.541 |
| 15 | hsa-miR-17_st | 0.0026 | 1.503 |
| 16 | hsa-miR-125a-5p_st | 0.00018 | −1.570 |
| 17 | hsa-miR-152_st | 0.00067 | −1.604 |
| 18 | hsa-miR-145_st | 0.00018 | −1.612 |
| 19 | hsa-miR-133b_st | 0.00022 | −1.952 |
| 20 | hsa-miR-455-3p_st | 0.00018 | −1.989 |
| 21 | hsa-miR-31_st | 0.00067 | −2.092 |
| 22 | hsa-miR-133a_st | 0.00012 | −2.219 |
| 23 | hsa-miR-222_st | 0.0001 | −2.251 |
| 24 | hsa-miR-221_st | 0.0001 | −2.261 |
| 25 | hsa-miR-205_st | 0.00005 | −3.867 |

TABLE 3
Table 3, List of examined CpG sites at the miR-31 promoter

| Region 1 | Region 2 | Region 3 | Region 4 |
|---|---|---|---|
| CpG_1 | CpG_1 | CpG_1.2 | CpG_1 |
| CpG_2.3 | CpG_2.3 | CpG_3.4.5 | CpG_2 |
| CpG_4 | CpG_4 | CpG_6 | CpG_4 |
| CpG_5 | CpG_5.6.7 | CpG_15 | CpG_5.6 |
| CpG_7 | CpG_16 | CpG_16.17.18 | CpG_7 |
| CpG_9.10.11.12 | CpG_17.18 | CpG_19 | CpG_8 |
| CpG_13.14.15.16 | CpG_19.20 | CpG_20.21 | CpG_9 |
| CpG_17.18 | CpG_31 | CpG_29 | CpG_10.11 |
| CpG_19.20.21 | CpG_32 | CpG_30 | |
| CpG_22.23 | CpG_36 | CpG_31 | |
| CpG_24.25.26.27.28 | | CpG_36 | |
| CpG_29.30 | | | |
| CpG_31 | | | |
| CpG_32 | | | |
| CpG_33 | | | |
| CpG_34 | | | |
| CpG_35.36 | | | |
| CpG_37 | | | |

TABLE 4
Table 4.1 Overall Difference of DNA Methylation levels
(B-T, n = 11 pairs)

| | | | Std | | |
|---|---|---|---|---|---|
| Minimum | Maximum | Mean | Dev | Median | p-value |
| −0.13 | −0.02 | −0.07 | 0.04 | −0.07 | 0.001 |

Table 4.2 Regional Difference of DNA Methylation levels
(B-T, n = 11 pairs)

| | | | | Std | | Adjusted |
|---|---|---|---|---|---|---|
| Region | Minimum | Maximum | Mean | Dev | Median | p-value |
| 1 | −0.2 | 0.02 | −0.1 | 0.07 | −0.12 | 0.0059 |
| 2 | −0.11 | 0 | −0.05 | 0.04 | −0.04 | 0.0059 |
| 3 | −0.08 | 0.01 | −0.04 | 0.03 | −0.05 | 0.0059 |
| 4 | −0.27 | 0.01 | −0.09 | 0.09 | −0.05 | 0.0039 |

Table 4.3 CpG units (B-T, n = 11 pairs)

| Region | CpG site | Adjusted p-value |
|---|---|---|
| 1 | CpG_9.10.11.12 | 0.0459 |
| 1 | CpG_19.20.21 | 0.0459 |
| 1 | CpG_22.23 | 0.0459 |

TABLE 5
Table 5.1 Overall DNA Methylation levels

| | Minimum | Maximum | Mean | Std Dev | Median | p-value |
|---|---|---|---|---|---|---|
| Gleason Scores = 6 | 0.04 | 0.12 | 0.06 | 0.02 | 0.05 | <0.0001 |
| Gleason Scores ≧ 7 | 0.05 | 0.3 | 0.13 | 0.06 | 0.12 | 0.0009 |
| METs | 0.26 | 0.43 | 0.31 | 0.07 | 0.29 | |

Table 5.2 Regional DNA Methylation levels

| | | | | | | | Adjusted |
|---|---|---|---|---|---|---|---|
| Region | | Minimum | Maximum | Mean | Std Dev | Median | p-value |
| 1 | Gleason Scores 6 | 0.06 | 0.23 | 0.09 | 0.05 | 0.07 | <0.0001 |
| | Gleason Scores ≧ 7 | 0.08 | 0.35 | 0.19 | 0.08 | 0.18 | 0.0114 |
| | METs | 0.22 | 0.6 | 0.43 | 0.14 | 0.45 | |
| 2 | Gleason Scores 6 | 0.03 | 0.06 | 0.04 | 0.01 | 0.04 | <0.0001 |
| | Gleason Scores ≧ 7 | 0.04 | 0.27 | 0.11 | 0.07 | 0.08 | 0.0162 |
| | METs | 0.14 | 0.44 | 0.24 | 0.13 | 0.2 | |
| 3 | Gleason Scores 6 | 0.02 | 0.04 | 0.03 | 0.01 | 0.03 | 0.0005 |
| | Gleason Scores ≧ 7 | 0.02 | 0.24 | 0.07 | 0.05 | 0.06 | 0.1944 |
| | METs | 0.03 | 0.2 | 0.13 | 0.08 | 0.16 | |
| 4 | Gleason Scores 6 | 0.03 | 0.07 | 0.05 | 0.01 | 0.04 | 0.0002 |
| | Gleason Scores ≧ 7 | 0.03 | 0.31 | 0.12 | 0.08 | 0.09 | 0.0114 |
| | METs | 0.16 | 0.71 | 0.37 | 0.22 | 0.36 | |

Table 5.3 CpG units
Gleason Score = 6 vs. Gleason Score ≧ 7 and METs

| | | Adjusted | | | Adjusted |
|---|---|---|---|---|---|
| Region | CpG site | p-value | Primer | CpG site | p-value |
| 1 | CpG_19.20.21 | 0.0007 | 1 | CpG_17.18 | 0.0191 |
| 1 | CpG_4 | 0.0011 | 2 | CpG_1 | 0.0191 |
| 1 | CpG_24.25.26.27.28 | 0.0021 | 2 | CpG_5.6.7 | 0.0191 |
| 4 | CpG_1 | 0.0045 | 1 | CpG_37 | 0.0202 |
| 1 | CpG_1 | 0.0048 | 3 | CpG_16.17.18 | 0.0202 |
| 1 | CpG_9.10.11.12 | 0.0056 | 1 | CpG_13.14.15.16 | 0.034 |
| 1 | CpG_5 | 0.0074 | 3 | CpG_31 | 0.035 |
| 4 | CpG_2 | 0.0113 | 1 | CpG_29.30 | 0.0437 |
| 2 | CpG_31 | 0.0122 | 2 | CpG_16 | 0.0462 |
| 1 | CpG_2.3 | 0.0124 | 2 | CpG_17.18 | 0.0462 |
| 2 | CpG_36 | 0.0182 | 2 | CpG_32 | 0.0462 |

Table 5.4 CpG units
Gleason Score ≧ 7 vs. METs

| Region | CpG site | Adjusted p-value |
|---|---|---|
| 1 | CpG_24.25.26.27.28 | 0.0234 |
| 2 | CpG_36 | 0.0322 |
| 4 | CpG_1 | 0.0477 |

TABLE 6

| Gene ontology | p-value |
|---|---|
| GO:0007049~Cell cycle | 1.15E−30 |
| GO:0007067~Mitosis | 5.60E−21 |
| GO:0006260~DNA replication | 3.33E−12 |
| GO:0007017~Microtubule-based process | 1.96E−10 |
| GO:0006281~DNA repair | 2.03E−06 |

TABLE 7

| | Forward strand | | Reverse strand | | Reverse strand | | Reverse strand |
|---|---|---|---|---|---|---|---|
| | Genomic | | Genomic | | Genomic | | Ggenomic |
| | locations of | | locations of | | locations of | | locations of |
| | Cytosines | | Cytosines | | Cytosines | | Cytosines |
| | (base pair) | | (base pair) | | (base pair) | | (base pair) |
| Region 1 | chr9: | Region 2 | chr9: | Region 3 | chr9: | Region 4 | chr9: |
| CpG_1 | 21,559,134 | CpG_1 | 21559735 | CpG_1.2 | 21560005, | CpG_1 | 21560120 |
| | | | | | 21560001 | | |
| CpG_2.3 | 21559147, | CpG_2.3 | 21559714, | CpG_3.4.5 | 21559976, | CpG_2 | 21560105 |
| | 21559154 | | 21559710 | | 21559972, | | |
| | | | | | 21559966, | | |
| CpG_4 | 21559162 | CpG_4 | 21559700 | CpG_6 | 21559958 | CpG_4 | 21560077 |
| CpG_5 | 21559190 | CpG_5.6.7 | 21559689, | CpG_15 | 21559900 | CpG_5.6 | 21560068, |
| | | | 21559685, | | | | 21560065 |
| | | | 21559682 | | | | |
| CpG_7 | 21559224 | CpG_16 | 21559637 | CpG_16.17.18 | 21559893, | CpG_7 | 21560046 |
| | | | | | 21559887, | | |
| | | | | | 21559884 | | |
| CpG_9.10.11.12 | 21559263, | CpG_17.18 | 21559623, | CpG_19 | 21559877 | CpG_8 | 21560040 |
| | 21559266, | | 21559615 | | | | |
| | 21559272, | | | | | | |
| | 21559274 | | | | | | |
| CpG_13.14.15.16 | 21559288, | CpG_19.20 | 21559597, | CpG_20.21 | 21559871, | CpG_9 | 21560030 |
| | 21559292, | | 21559589 | | 21559866 | | |
| | 21559295, | | | | | | |
| | 21559300 | | | | | | |
| CpG_17.18 | 21559307, | CpG_31 | 21559498 | CpG_29 | 2559816 | CpG_10.11 | 21560005, |
| | 21559310 | | | | | | 21560001 |
| CpG_19.20.21 | 21559317, | CpG_32 | 21559492 | CpG_30 | 21559808 | | |
| | 21559319, | | | | | | |
| | 21559322 | | | | | | |
| CpG_22.23 | 21559330, | CpG_36 | 21559437 | CpG_31 | 21559779 | | |
| | 21559338 | | | | | | |
| CpG_24.25.26.27.28 | 21559344, | | | CpG_36 | 21559667 | | |
| | 21559348, | | | | | | |
| | 21559351, | | | | | | |
| | 21559354, | | | | | | |
| | 21559361 | | | | | | |
| CpG_29.30 | 21559367, | | | | | | |
| | 21559374 | | | | | | |
| CpG_31 | 21559381 | | | | | | |
| CpG_32 | 21559395 | | | | | | |
| CpG_33 | 21559410 | | | | | | |
| CpG_34 | 21559436 | | | | | | |
| CpG_35.36 | 21559456, | | | | | | |
| | 21559758 | | | | | | |
| CpG_37 | 21559463 | | | | | | |

1. A method of diagnosing prostate cancer in a subject comprising
(a) obtaining a biological sample from said subject, and
(b) measuring the level of miR-31 promoter methylation in said sample, and
(c) detecting an alteration in the level of miR-31 promoter methylation, wherein detection of an alteration in the level of miR-31 promoter methylation indicates the presence of said prostate cancer in said subject.

2. The method of claim 1, wherein said sample is selected from the group consisting of whole blood, urine, tissue, lymph node or a combination thereof.

3. The method of claim 1, wherein the level of miR-31 indicates the severity of prostate cancer in said subject.

4. The method of claim 1, further comprising comparing the level of miR-31 promoter methylation in said sample to that of a sample obtained from benign tissue.

5. The method of claim 4, wherein the benign tissue is benign prostate tissue.

6. The method of claim 1, wherein the level of miR-31 promoter methylation is measured by a process selected from, methylation-specific polymerase chain reaction, single-molecule, real-time sequencing, bisulfite DNA sequencing, HPLC, mass spectrometry, microarray or methylated DNA immunoprecipitation.

7. The method of claim 6, wherein said process is mass spectrometry of bisulfite treated DNA of miR-31.

8. The method of claim 1, wherein the level of miR-31 promoter methylation is measured by a process comprising polymerase chain reaction in which miR-31 DNA is amplified using PCR primers selected from the group consisting of SEQ ID NOS: 2-9.

9. A method of diagnosing prostate cancer in a subject comprising
(a) obtaining a biological sample from said subject, and
(b) measuring the level of expression of miR-31 in said biological sample, wherein the level of miR-31 indicates the presence of prostate cancer in said subject.

10. The method of claim 9, wherein said biological sample is selected from the group consisting of whole blood, urine, tissue, lymph node or a combination thereof.

11. The method of claim 9, wherein the level of miR-31 indicates the severity of prostate cancer in said subject.

12. The method of claim 9, further comprising comparing the level of miR-31 expression in said biological sample to that of a second biological isolated from benign tissue.

13. The method of claim 12, wherein the benign tissue is benign prostate tissue.

14. A method of determining whether a subject is a candidate for treatment of prostate cancer with an AR targeting therapeutic agent comprising
(a) obtaining a biological sample from said subject, and
(b) measuring the level of miR-31 promoter methylation in said biological sample, and
(c) detecting an alteration in the level of miR-31 promoter methylation, wherein the subject is rejected as a candidate if the level of miR-31 promoter methylation is decreased or said subject is selected as a candidate if the level of miR-31 promoter methylation is increased.

15-17. (canceled)

18. A method of treating prostate cancer in a subject, comprising administering to the subject an effective amount of an agent that modulates the activity of miR-31.

19-37. (canceled)

38. An isolated nucleic acid used for modulating the activity of AR in a cell that is identical to or substantially identical to the nucleotide sequence UCGAUACGGUCGUAGAACGGA [SEQ ID NO:1].


SEQUENCE LISTING (109 sequences)

SEQ ID NO: 1 - RNA, 21 nt, Artificial Sequence
  microRNA-31 nucleotide sequence
ucgauacggucguagaacgga

SEQ ID NO: 2 - DNA, 25 bp, Artificial Sequence
  Forward primer for amplifying CpG island 1 in the miR-31 promoter region
ttttttttaagaagggaaagtttag

SEQ ID NO: 3 - DNA, 27 bp, Artificial Sequence
  Reverse primer for amplifying CpG island 1 in the miR-31 promoter region
caaataaactaaaaaaaccttaatccc

SEQ ID NO: 4 - DNA, 25 bp, Artificial Sequence
  Forward primer for amplifying CpG island 2 in the miR-31 promoter region
tttttaggaggagtttggtgagtag

SEQ ID NO: 5 - DNA, 21 bp, Artificial Sequence
  Reverse primer for amplifying CpG island 2 in the miR-31 promoter region
aacctcctcaactccttaaaa

SEQ ID NO: 6 - DNA, 24 bp, Artificial Sequence
  Forward primer for amplifying CpG island 3 in the miR-31 promoter region
gttgtagtggagaaatttgggttt

SEQ ID NO: 7 - DNA, 20 bp, Artificial Sequence
  Reverse primer for amplifying CpG island 3 in the miR-31 promoter region
caccaaactcctcctaaaaa

SEQ ID NO: 8 - DNA, 24 bp, Artificial Sequence
  Forward primer for amplifying CpG island 4 in the miR-31 promoter region
tggtttttgtaggtggattttttt

SEQ ID NO: 9 - DNA, 24 bp, Artificial Sequence
  Reverse primer for amplifying CpG island 4 in the miR-31 promoter region
tactaaacctcctcccttaaaccc

SEQ ID NO: 10 - DNA, 20 bp, Artificial Sequence
  Forward primer for PCR of MIR31HG promoter region
ccccaagttatgcacaggtc

SEQ ID NO: 11 - DNA, 20 bp, Artificial Sequence
  Reverse primer for PCR of MIR31HG promoter region
cccttcaaatccaggtgaaa

SEQ ID NO: 12 - DNA, 20 bp, Artificial Sequence
  Forward primer for PCR of MIR31HG promoter region
taaagcagctgcccaatttt

SEQ ID NO: 13 - DNA, 20 bp, Artificial Sequence
  Reverse primer for PCR of MIR31HG promoter region
cgaagtcacaggttcgctct

SEQ ID NO: 14 - DNA, 31 bp, Artificial Sequence
  T7 promoter sequence for binding of T7 polymerase during transcription and cloning into vectors
cagtaatacgactcactatagggagaaggct

SEQ ID NO: 15 - RNA, 21 nt, Artificial Sequence
  Therapeutic nucleic acid corresponding to a portion of miR-31 that binds AR
ucgnnncngucnuagaacngn

SEQ ID NO: 16 - RNA, 23 nt, Artificial Sequence
  Therapeutic nucleic acid corresponding to a portion of miR-31 that binds AR
ucnanncggunncgnngaacgga

SEQ ID NO: 17 - RNA, 27 nt, Artificial Sequence
  Therapeutic nucleic acid corresponding to a portion of miR-31 that binds AR
nnnnnncggucnnnnnnnnanaacggn

SEQ ID NO: 18 - DNA, 34 bp, Artificial Sequence
  Forward primer for miR-31 CpG region 3
aggaagagaggttgtagtggagaaatttgggttt

SEQ ID NO: 19 - DNA, 53 bp, Artificial Sequence
  Reverse primer for miR-31 CpG region 3
cagtaatacgactcactatagggagaaggctctcaccaaactcctcctaaaaa

SEQ ID NO: 20 - DNA, 34 bp, Artificial Sequence
  Forward primer for miR-31 CpG region 4
aggaagagagtggtttttgtaggtggattttttt

SEQ ID NO: 21 - DNA, 55 bp, Artificial Sequence
  Reverse primer for miR-31 CpG region 4
cagtaatacgactcactatagggagaaggcttactaaacctcctcccttaaaccc

SEQ ID NO: 22 - DNA, 20 bp, Artificial Sequence
  Forward primer for PCR of MIR31HG mRNA
cgcttctgtcctcctactcg

SEQ ID NO: 23 - DNA, 20 bp, Artificial Sequence
  Reverse primer for PCR of MIR31HG mRNA
acaagcagacccttggaatg

SEQ ID NO: 24 - DNA, 18 bp, Artificial Sequence
  Forward primer for PCR of PSA mRNA
tgtgtgctggacgctgga

SEQ ID NO: 25 - DNA, 19 bp, Artificial Sequence
  Reverse primer for PCR of PSA mRNA
cactgccccatgacgtgat

SEQ ID NO: 26 - DNA, 22 bp, Artificial Sequence
  Forward primer for PCR of TMPRSS2 mRNA
ggacagtgtgcacctcaaagac

SEQ ID NO: 27 - DNA, 18 bp, Artificial Sequence
  Reverse primer for PCR of TMPRSS2 mRNA
tcccacgaggaaggtccc

SEQ ID NO: 28 - DNA, 20 bp, Artificial Sequence
  Forward primer for PCR of NDRG1 mRNA
gtggagaaaggggagaccat

SEQ ID NO: 29 - DNA, 20 bp, Artificial Sequence
  Reverse primer for PCR of NDRG1 mRNA
acagcgtgacgtgaacagag

SEQ ID NO: 30 - DNA, 20 bp, Artificial Sequence
  Forward primer for PCR of HMBS mRNA
ccatcatcctggcaacagct

SEQ ID NO: 31 - DNA, 19 bp, Artificial Sequence
  Reverse primer for PCR of HMBS mRNA
gcattcctcagggtgcagg

SEQ ID NO: 32 - DNA, 20 bp, Artificial Sequence
  Forward primer for miR-31 promoter region 1000 nucleotides downstream of miR-31HG gene start site
ccgatgacctagccagaagt

SEQ ID NO: 33 - DNA, 20 bp, Artificial Sequence
  Reverse primer for miR-31 promoter region 1000 nucleotides downstream of miR-31HG gene start site
ccccacccttcaactcgtag

SEQ ID NO: 34 - DNA, 20 bp, Artificial Sequence
  Forward primer for miR-31 promoter region 500 nucleotides downstream of miR-31HG gene start site
tatcctcaaccctccgtgtc

SEQ ID NO: 35 - DNA, 20 bp, Artificial Sequence
  Reverse primer for miR-31 promoter region 500 nucleotides downstream of miR-31HG gene start site
catacacctgaaggggcagt

SEQ ID NO: 36 - DNA, 20 bp, Artificial Sequence
  Forward primer for miR-31 promoter region 1000 nucleotides upstream of miR-31HG gene start site
caattttggcccaggagata

SEQ ID NO: 37 - DNA, 20 bp, Artificial Sequence
  Reverse primer for miR-31 promoter region 500 nucleotides upstream of miR-31HG gene start site
tttccggggacctctagttt

SEQ ID NO: 38 - DNA, 24 bp, Artificial Sequence
  Forward primer for miR-31 promoter region 42,500 nucleotides upstream of miR-31HG gene start site
tggcctatttgctgttctaatgac

SEQ ID NO: 39 - DNA, 17 bp, Artificial Sequence
  Reverse primer for miR-31 promoter region 42,500 nucleotides upstream of miR-31HG gene start site
gcaagccaaccccaaca

SEQ ID NO: 40 - DNA, 19 bp, Artificial Sequence
  Forward primer for miR-31 promoter region 45,000 nucleotides upstream of miR-31HG gene start site
aatgggccctgcattctct

SEQ ID NO: 41 - DNA, 20 bp, Artificial Sequence
  Reverse primer for miR-31 promoter region 45000 nucleotides upstream of miR-31HG gene start site
aaaacccacaccctcaccac

SEQ ID NO: 42 - DNA, 22 bp, Artificial Sequence
  Forward primer for miR-31 promoter region 47,500 nucleotides upstream of miR-31HG gene start site
catcttcaaaagcggacactct

SEQ ID NO: 43 - DNA, 23 bp, Artificial Sequence
  Reverse primer for miR-31 promoter region 47,500 nucleotides upstream of miR-31HG gene start site
acaatacatagcaggacaggaag

SEQ ID NO: 44 - DNA, 26 bp, Artificial Sequence
  Forward primer for PSA gene start site
cctagatgaagtctccatgagctaca

SEQ ID NO: 45 - DNA, 21 bp, Artificial Sequence
  Reverse primer for PSA gene start site
gggagggagagctagcacttg

SEQ ID NO: 46 - DNA, 20 bp, Artificial Sequence
  Forward primer for MYT1 gene start site
aggcaccttctgttggccga

SEQ ID NO: 47 - DNA, 20 bp, Artificial Sequence
  Reverse primer for MYT1 gene start site
aggcagctgcctcccgtaca

SEQ ID NO: 48 - DNA, 20 bp, Artificial Sequence
  Forward primer for GAPDH gene start site
cggctactagcggttttacg

SEQ ID NO: 49 - DNA, 20 bp, Artificial Sequence
  Reverse primer for GAPDH gene start site
aagaagatgcggctgactgt

SEQ ID NO: 50 - DNA, 28 bp, Artificial Sequence
  Forward primer for construct preparation of AR microRNA recognition element 1
tcgagctagcagggcagatcttgtccgc

SEQ ID NO: 51 - DNA, 28 bp, Artificial Sequence
  Reverse primer for construct preparation of AR microRNA recognition element 1
ggccgcggacaagatctgccctgctagc

SEQ ID NO: 52 - DNA, 28 bp, Artificial Sequence
  Forward primer for construct preparation of AR microRNA recognition element 2
tcgagaagtgggccaaggccttgcctgc

SEQ ID NO: 53 - DNA, 28 bp, Artificial Sequence
  Reverse primer for construct preparation of AR microRNA recognition element 2
ggccgcaggcaaggccttggcccacttc

SEQ ID NO: 54 - DNA, 28 bp, Artificial Sequence
  Forward primer for construct preparation of AR microRNA recognition element 3
tcgaggccagggaccatgttttgcccgc

SEQ ID NO: 55 - DNA, 28 bp, Artificial Sequence
  Reverse primer for construct preparation of AR microRNA recognition element 3
ggccgcgggcaaaacatggtccctggcc

SEQ ID NO: 56 - DNA, 28 bp, Artificial Sequence
  Forward primer for construct preparation of AR microRNA recognition element 4
tcgagatctgttccatcttcttgcctgc

SEQ ID NO: 57 - DNA, 28 bp, Artificial Sequence
  Reverse primer for construct preparation of AR microRNA recognition element 4
ggccgcaggcaagaagatggaacagatc

SEQ ID NO: 58 - DNA, 28 bp, Artificial Sequence
  Forward primer for construct preparation of the AR microRNA recognition element 2 mutant (mt)
tcgagaagtgggccaaggccaagggtgc

SEQ ID NO: 59 - DNA, 28 bp, Artificial Sequence
  Reverse primer for construct preparation of the AR microRNA recognition element 2 mutant (mt)
ggccgcacccttggccttggcccacttc

SEQ ID NO: 60 - DNA, 28 bp, Artificial Sequence
  Forward primer for construct preparation of the AR microRNA recognition element 2 mutant (mt1)
tcgagaagtgggccgaggccttgcctgc

SEQ ID NO: 61 - DNA, 28 bp, Artificial Sequence
  Reverse primer for construct preparation of the AR microRNA recognition element 2 mutant (mt1)
ggccgcaggcaaggcctcggcccacttc

SEQ ID NO: 62 - DNA, 28 bp, Artificial Sequence
  Forward primer for construct preparation of the AR microRNA recognition element 2 mutant (mt2)
tcgagaagtgggccaagaccttgcctgc

SEQ ID NO: 63 - DNA, 28 bp, Artificial Sequence
  Reverse primer for construct preparation of the AR microRNA recognition element 2 mutant (mt2)
ggccgcaggcaaggtcttggcccacttc

SEQ ID NO: 64 - DNA, 28 bp, Artificial Sequence
  Forward primer for construct preparation of the AR microRNA recognition element 2 mutant (mt3)
tcgagaagtgggccaaggcctttcctgc

SEQ ID NO: 65 - DNA, 28 bp, Artificial Sequence
  Reverse primer for construct preparation of the AR microRNA recognition element 2 mutant (mt3)
ggccgcaggaaaggccttggcccacttc

SEQ ID NO: 66 - DNA, 27 bp, Artificial Sequence
  Forward primer for construct preparation of the AR microRNA recognition element 3 mutant (mt4)
tcgaggccagggaccatgttttcccgc

SEQ ID NO: 67 - DNA, 27 bp, Artificial Sequence
  Reverse primer for construct preparation of the AR microRNA recognition element 3 mutant (mt4)
ggccgcgggaaaacatggtccctggcc

SEQ ID NO: 68 - DNA, 28 bp, Artificial Sequence
  Forward primer for construct preparation of the AR microRNA recognition element 4 mutant (mt)
tcgagatctgttccatcttgaagggtgc

SEQ ID NO: 69 - DNA, 28 bp, Artificial Sequence
  Reverse primer for construct preparation of the AR microRNA recognition element 4 mutant (mt)
ggccgcacccaacaagatggaacagatc

SEQ ID NO: 70 - DNA, 30 bp, Artificial Sequence
  Forward primer for construct preparation of the CDK1 3′ UTR domain
atgactcgagacgaatttctggcaaaatgg

SEQ ID NO: 71 - DNA, 33 bp, Artificial Sequence
  Reverse primer for construct preparation of the CDK1 3′ UTR domain
atgagcggccgcgagcctttttagatggctgct

SEQ ID NO: 72 - DNA, 30 bp, Artificial Sequence
  Forward primer for construct preparation of the E2F2 3′UTR domain
atgactcgagccccctctactgtccctttc

SEQ ID NO: 73 - DNA, 32 bp, Artificial Sequence
  Reverse primer for construct preparation of the E2F2 3′UTR domain
atgagcggccgcggaccatgttctctcggttc

SEQ ID NO: 74 - DNA, 30 bp, Artificial Sequence
  Forward primer for construct preparation of the EXO1 3′UTR domain
atgactcgagtccagaagcggaagaggata

SEQ ID NO: 75 - DNA, 33 bp, Artificial Sequence
  Reverse primer for construct preparation of the EXO1 3′UTR domain
atgagcggccgccctccaaaaacctgtcagaga

SEQ ID NO: 76 - DNA, 31 bp, Artificial Sequence
  Forward primer for construct preparation of the FOXM1 3′UTR domain
atgactcgagccctgacaacatcaactggtc

SEQ ID NO: 77 - DNA, 32 bp, Artificial Sequence
  Reverse primer for construct preparation of the FOXM1 3′UTR domain
atgagcggccgctttttgtccaccttcgcttt

SEQ ID NO: 78 - DNA, 30 bp, Artificial Sequence
  Forward primer for construct preparation of the MCM2 3′UTR domain
atgactcgagttccttgggattctggtttg

SEQ ID NO: 79 - DNA, 32 bp, Artificial Sequence
  Reverse primer for construct preparation of the MCM2 3′UTR domain
atgagcggccgccagcctgaatacgcaactca

SEQ ID NO: 80 - DNA, 30 bp, Artificial Sequence
  Forward primer for construct preparation of the AR long 3′ UTR domain
atgactcgagtggagccagaggagaagaaa

SEQ ID NO: 81 - DNA, 32 bp, Artificial Sequence
  Reverse primer for construct preparation of the AR long 3′ UTR domain
atgagcggccgcgagttcatgggtggcaaagt

SEQ ID NO: 82 - DNA, 28 bp, Artificial Sequence
  Forward primer for construct preparation of the CDK1 microRNA recognition element
cgagagagcatgccaaaatttgctaagc

SEQ ID NO: 83 - DNA, 29 bp, Artificial Sequence
  Reverse primer for construct preparation of the CDK1 microRNA recognition element
ggccgcttagcaaattttggcatgctctc

SEQ ID NO: 84 - DNA, 29 bp, Artificial Sequence
  Forward primer for construct preparation of the E2F2 microRNA recognition element 1
tcgagagctcatgcccccgtcttgccagc

SEQ ID NO: 85 - DNA, 29 bp, Artificial Sequence
  Reverse primer for construct preparation of the E2F2 microRNA recognition element 1
ggccgctggcaagacgggggcatgagctc

SEQ ID NO: 86 - DNA, 29 bp, Artificial Sequence
  Forward primer for construct preparation of the E2F2 microRNA recognition element 1 mutant
tcgagacgactacggtcgtagaacggagc

SEQ ID NO: 87 - DNA, 29 bp, Artificial Sequence
  Reverse primer for construct preparation of the E2F2 microRNA recognition element 1 mutant
ggccgctccgttctacgaccgtagtcgtc

SEQ ID NO: 88 - DNA, 28 bp, Artificial Sequence
  Forward primer for construct preparation of the E2F2 microRNA recognition element 2
tcgaggtgagctgaagaaccttgcctgc

SEQ ID NO: 89 - DNA, 28 bp, Artificial Sequence
  Reverse primer for construct preparation of the E2F2 microRNA recognition element 2
ggccgcaggcaaggttcttcagctcacc

SEQ ID NO: 90 - DNA, 28 bp, Artificial Sequence
  Forward primer for construct preparation of the E2F2 microRNA recognition element 3
tcgaggcagctgtgggcccttttgccgc

SEQ ID NO: 91 - DNA, 28 bp, Artificial Sequence
  Reverse primer for construct preparation of the E2F2 microRNA recognition element 3
ggccgcggcaaaagggcccacagctgcc

SEQ ID NO: 92 - DNA, 29 bp, Artificial Sequence
  Forward primer for construct preparation of the FOXM1 microRNA recognition element
tcgaggacaagtggatctgcttgccaggc

SEQ ID NO: 93 - DNA, 29 bp, Artificial Sequence
  Reverse primer for construct preparation of the FOXM1 microRNA recognition element
ggccgcctggcaagcagatccacttgtcc

SEQ ID NO: 94 - DNA, 29 bp, Artificial Sequence
  Forward primer for construct preparation of the FOXM1 microRNA recognition element mutant
tcgaggagatgtggttgtggaacggaggc

SEQ ID NO: 95 - DNA, 29 bp, Artificial Sequence
  Reverse primer for construct preparation of the FOXM1 microRNA recognition element mutant
ggccgcctccgttccacaaccacatctcc

SEQ ID NO: 96 - DNA, 28 bp, Artificial Sequence
  Forward primer for construct preparation of the MCM2 microRNA recognition element
tcgagtggttgctgaacatcttgccagc

SEQ ID NO: 97 - DNA, 28 bp, Artificial Sequence
  Reverse primer for construct preparation of the MCM2 microRNA recognition element
ggccgctggcaagatgttcagcaaccac

SEQ ID NO: 98 - DNA, 28 bp, Artificial Sequence
  Forward primer for construct preparation of the MCM2 microRNA recognition element mutant
tcgagtggttgctgaacatcaagggagc

SEQ ID NO: 99 - DNA, 28 bp, Artificial Sequence
  Reverse primer for construct preparation of the MCM2 microRNA recognition element mutant
ggccgctcccttgatgttcagcaaccac

SEQ ID NO: 100 - DNA, 37 bp, Artificial Sequence
  Forward primer for site directed mutagenesis of the AR long 3′UTR mutant
ggcaagctgggcgtcttaaacttgtcccccagagatg

SEQ ID NO: 101 - DNA, 37 bp, Artificial Sequence
  Reverse primer for site directed mutagenesis of the AR long 3′UTR mutant
catctctgggggacaagtttaagacgcccagcttgcc

SEQ ID NO: 102 - DNA, 38 bp, Artificial Sequence
  Forward primer for site directed mutagenesis of the AR CDS mutant
caagtgggccaaggcctttcctggcttccgcaacttac

SEQ ID NO: 103 - DNA, 38 bp, Artificial Sequence
  Reverse primer for site directed mutagenesis of the AR CDS mutant
gtaagttgcggaagccaggaaaggccttggcccacttg

SEQ ID NO: 104 - DNA, 40 bp, Artificial Sequence
  Forward primer for site directed mutagenesis of the E2F1 3′UTR mutant
gtttccagagatgctcaccaactctctgcagccctggagc

SEQ ID NO: 105 - DNA, 40 bp, Artificial Sequence
  Reverse primer for site directed mutagenesis of the E2F1 3′UTR mutant
gctccagggctgcagagagttggtgagcatctctggaaac

SEQ ID NO: 106 - DNA, 35 bp, Artificial Sequence
  Forward primer for miR-31 CpG region1
aggaagagagttttttttaagaagggaaagtttag

SEQ ID NO: 107 - DNA, 58 bp, Artificial Sequence
  Reverse primer for miR-31 CpG region1
cagtaatacgactcactatagggagaaggctcaaataaactaaaaaaaccttaatccc

SEQ ID NO: 108 - DNA, 35 bp, Artificial Sequence
  Forward primer for miR-31 CpG region 2
aggaagagagtttttaggaggagtttggtgagtag

SEQ ID NO: 109 - DNA, 53 bp, Artificial Sequence
  Reverse primer for miR-31 CpG region 2
cagtaatacgactcactatagggagaaggctaaacctcctcaactccttaaaa